(19) United States
(12) Patent Application Publication
(10) Pub. No.: US 2026/0015586 A1
(43) Pub. Date: Jan. 15, 2026

(54) POPULATION-SCALE ORGANOID POOLS

(71) Applicant: Children's Hospital Medical Center, Cincinnati, OH (US)

(72) Inventors: Takanori TAKEBE, Cincinnati, OH (US); Masaki KIMURA, Cincinnati, OH (US)

(73) Assignee: Children's Hospital Medical Center, Cincinnati, OH (US)

(21) Appl. No.: 18/995,067

(22) PCT Filed: Jul. 21, 2023
(86) PCT No.: PCT/US2023/028409

§ 371 Date: Jan. 15, 2025

Related U.S. Application Data
(60) Provisional application No. 63/393,728, filed on Jul. 29, 2022.

(51) Int. Cl. C12N 5/071; A61K 31/155; A61K 31/426; A61K 31/706; A61P 1/16; C12Q 1/6883

(52) U.S. Cl. CPC C12N 5/0671; A61K 31/155; A61K 31/426; A61K 31/706; A61P 1/16; C12Q 1/6883; C12N 2500/38; C12N 2501/115; C12N 2501/119; C12N 2501/12; C12N 2501/15; C12N 2501/155; C12N 2501/16; C12N 2501/237; C12N 2501/39; C12N 2501/415; C12N 2503/04; C12N 2506/45; C12Q 2600/156

(57) ABSTRACT

Disclosed are improved methods of making liver organoids, and methods of making population organoid panels which can be used, for example, for genotype-phenotype analysis or screening of test compounds. Also disclosed are methods of assessing the risk/prognosis of fatty acid liver disease in subjects having a SNP variant GCKR-rs1260326, as well as novel methods of treating such subjects.

### CROSS-REFERENCE TO RELATED APPLICATIONS

This application claims priority to and benefit of U.S. Provisional Patent Application No. 63/393,728, filed Jul. 29, 2022.

### STATEMENT REGARDING FEDERALLY SPONSORED R&D

This invention was made with government support under DP2 DK128799-01 and UG3 DK119982 awarded by the National Institutes of Health. The government has certain rights to the invention.

### FIELD OF THE INVENTION

Aspects of the present disclosure relate generally to methods of generating a population organoid panel, for example liver organoids. Additional aspects relate to the generation of steatohepatitis models. Additional aspects relate to diagnosing and treating subjects at increased risk of and/or having a poor prognosis for fatty acid liver disease.

### BACKGROUND

Although our understanding of the genetic underpinnings in many diseases have advanced, known risk variants explain only a modest fraction of heritability in common disorders such as the metabolic non-alcoholic fatty liver disease (NAFLD), despite genetic association studies in monozygotic twins. Genetic pleiotropy, when intersected with metabolic traits and disorders, can further complicate genetic interpretation of pathogenicity. Worldwide, NAFLD is now one of the most common chronic liver diseases affecting nearly 25% of the adult population. Type 2 diabetes (T2D), itself a prevalent metabolic disease, is a major comorbidity of NAFLD. In the US, the co-prevalence of NAFLD and T2D has been reported to range from 32% to 90%, depending on the age group.

### SUMMARY

Exemplary embodiments of the present disclosure are provided in the following numbered embodiments:

1. A method of making a liver organoid comprising:
a) embedding a foregut progenitor (FG) cell in a basement membrane matrix environment;
b) exposing the embedded FG cell to an FGF activator, a TGF-beta inhibitor, and a Wnt pathway activator for a period sufficient to promote expansion of the FG cell;
c) exposing the expanded FG cells of b) to a retinoic acid pathway activator for a period of time sufficient to differentiate the expanded FG cells into a liver organoid; and
d) optionally exposing the liver organoid of c) to hepatocyte growth factor (HGF), oncostatin M (OSM), dexamethasone (DEX) and insulin for a period of time.

2. A method of making a population organoid panel comprising:
a) embedding a plurality of individual progenitor cells in a single basement membrane matrix environment, wherein each of the plurality of progenitor cells is from a different donor, and
b) differentiating the plurality of progenitor cells into organoids.

3. The method of embodiment 2, wherein the population organoid panel is a liver organoid panel, the progenitor cells are FG cells, and differentiating the plurality of FG cells into liver organoids comprises:
a) exposing the embedded FG cells to an FGF activator, a TGF-beta inhibitor, and a Wnt pathway activator for a period sufficient to promote expansion of the FG cells to form a plurality of expanded FG cells, wherein each of the plurality of expanded FG cells of comprises cells from only a single donor;
b) exposing the plurality of expanded FG cells to retinoic acid for a period of time sufficient to differentiate them into a plurality of liver organoids, wherein each liver organoid of the plurality of liver organoids comprises cells from only a single donor; and
c) optionally exposing the liver organoids of b) to hepatocyte growth factor (HGF), oncostatin M (OSM), dexamethasone (DEX) and insulin for a period of time.

4. The method of any one of embodiments 1-3, wherein the FGF activator is FGF2, optionally in an amount of 0.5-50 ng/ml, 1-25 ng/mL, 2.5-10 ng/mL, or 5 ng/mL.

5. The method of any one of embodiments 1-4, wherein the TGF-beta inhibitor is A83 01, optionally in an amount of 0.05-5.0 μM, 0.1-2.5 μM, 0.25-1.0 μM, or 0.5 μM.

6. The method of any one of embodiments 1-5, wherein the Wnt pathway activator is a GSK-3 inhibitor, optionally CHIR99021, optionally in an amount of 0.3-30 PM, 0.6-15 μM, 1.5-6 μM, or 3 μM.

7. The method of any one of embodiments 1-6, wherein the retinoic acid pathway activator is retinoic acid, optionally in an amount of 0.2-20 PM, 0.4-10 PM, 1.0-4 μM, or 2 μM.

8. The method of any one of embodiments 1-7, wherein the period sufficient to promote expansion of the FG cell to form expanded FG cells is about 1-8, 2-6, 3-5, or 4 days.

9. The method of any one of embodiments 1-8, wherein the period of time sufficient to differentiate the expanded FG cells into a liver organoid is about 1-8, 2-6, 3-5, or 4 days.

10. The method of any one of embodiments 1-9, further comprising culturing the liver organoid in hepatocyte culture medium.

11. The method of any one of embodiments 1-10, wherein the FG cell is differentiated from an induced-pluripotent stem cell (IPSC) by exposure to Activin A and optionally a BMP pathway activator, a Wnt pathway activator, and an FGF activator.

12. The method of embodiment 11, wherein the IPSC is exposed to Activin A and optionally a BMP pathway activator, optionally BMP4, for a first period of time, optionally about 1-4, 2-4, or 3 days, and then exposed to the Wnt pathway activator and an FGF activator for a second period of time, optionally about 1-4, 2-4, or 3 days.

13. The method of embodiment 11 or 12, wherein the method comprises dissociating clusters of FG cells into single FG cells, and optionally, cryopreserving single FG cells.

14. The method of any one of embodiments 11-13, wherein:
the Activin A is in an amount of 1-10000 ng/mL, 10-1000 ng/mL, 20-500 ng/mL, 50-200 ng/mL, or 100 ng/mL,
the Wnt pathway activator is a GSK-3 inhibitor, optionally CHIR99021, optionally in an amount of 0.3-30 μM, 0.6-15 μM, 1.5-6 μM, or 3 μM; and/or
the FGF activator is FGF4, optionally in an amount of 50-5000 ng/ml, 100-2500 ng/mL, 250-1000 ng/mL, or 500 ng/mL.

15. The method of any one of embodiments 2-14, wherein the plurality of individual FG cells are from at least 20 different donors.

16. The method of any one of embodiments 2-15, wherein the liver organoid has a genotype comprising a single nucleotide polymorphism (SNP) variant selected from the group consisting of PNPLA3 rs738409, GCKR-rs1260326, GCKR-rs780094, and TM6SF2-rs58542926.

17. The method of any one of embodiments 1-16, wherein the FG cell is human.

18. The method of any one of embodiments 1-17, further comprising exposing the liver organoid to a fatty acid, optionally oleic acid, and optionally insulin, to generate a steatohepatitis-like liver organoid.

19. A method of determining a genotype associated with a non-alcoholic fatty liver disease (NAFLD) or non-alcoholic steatohepatitis (NASH) phenotype, comprising
a) generating a population liver organoid panel with steatohepatitis-like liver organoids by the method of embodiment 18;
b) maintaining the population liver organoid panel in a shared environmental condition;
c) observing the phenotype of an individual steatohepatitis-like organoid;
d) sequencing a nucleic acid sample of the individual steatohepatitis-like clonal organoid to the identify a genotype of the individual steatohepatitis-like clonal organoid; and
e) correlating the genotype of the individual steatohepatitis-like clonal organoid with the observed phenotype of the individual steatohepatitis-like clonal organoid.

20. The method of any one of embodiments 3-19, further comprising administering a compound of interest to the population liver organoid panel.

21. The method of embodiment 20, wherein the compound of interest is selected based on a correlated genotype-phenotype of the steatohepatitis-like clonal organoid.

22. The method of embodiment 20 or 21, further comprising assessing the efficacy of the compound of interest for treating NAFLD/NASH by observing a response of the individual steatohepatitis-like clonal organoid to the compound.

23. The method of any one of embodiments 20-22, wherein the compound of interest is PFK15, AMG3969, metformin, nitazoxanide (NTZ), and/or nicotinamide riboside (NR).

24. The method of any one of embodiments 19-23 wherein the shared environmental conditions are insulin insensitive.

25. The method of embodiment 24, wherein the insulin insensitive conditions comprise high glucose and/or high insulin culture conditions.

26. The method of any one of embodiments 19-25, wherein the observed phenotype is lipid accumulation, inflammation and/or mitochondrial function

27. The method of any one of embodiments 19-26, wherein the genotype is a single nucleotide polymorphism (SNP) variant.

28. The method of embodiment 27, wherein the single nucleotide polymorphism (SNP) variant is PNPLA3 rs738409, GCKR-rs1260326, GCKR-rs780094, or TM6SF2-rs58542926.

29. A liver organoid made by the method of any one of embodiments 1-18.

30. A liver population organoid panel made by the method of any one of embodiments 3-18.

31. The liver population organoid panel of embodiment 30, wherein the organoids are liver organoids and/or steatohepatitis-like liver organoids.

32. A liver organoid having a SNP variant selected from the group consisting of PNPLA3 rs738409, GCKR-rs1260326, GCKR-rs780094, and TM6SF2-rs58542926.

33. A method of screening a compound of interest comprising administering a compound of interest to the liver organoid of embodiment 29 or 32, or the liver population organoid panel of embodiment 30 or 31.

34. The method of embodiment 33, wherein the compound of interest is selected based on a correlated genotype-phenotype of the steatohepatitis-like clonal organoid.

35. A method comprising:
a) obtaining or having obtained the HbA1c level of a subject identified as having a SNP variant GCKR-rs1260326;
b) determining that the subject has a reduced risk of and/or a good prognosis for fatty acid liver disease if the subject's HbA1c level is less than 5.7%, or
c) determining that the subject has an increased risk of and/or a poor prognosis for fatty acid liver disease if the subject's HbA1c level is greater than 6.4%.

36. A method for treating a subject identified as having a SNP variant GCKR-rs1260326, the method comprising:
a) obtaining or having obtained the HbA1c level of the subject identified as having a SNP variant GCKR-rs1260326;
b) determining that the subject has a reduced risk of and/or a good prognosis for fatty acid liver disease if the subject's HbA1c level is less than 5.7%, or determining that the subject has an increased risk of and/or a poor prognosis for fatty acid liver disease if the subject's HbA1c level is greater than 6.4%; and
c) administering to the subject that has an increased risk of fatty acid liver disease having an HbA1c level greater than 6.4% a treatment that results in oxidative uncoupling.

37. A method for treating a subject comprising:
selecting a subject identified as having a SNP variant GCKR-rs1260326 and HbA1c level greater than 6.4%; and
administering to the subject a treatment that results in oxidative uncoupling.

38. The method of embodiment 36 or 37, wherein treatment that results in oxidative uncoupling comprises a NAD+ precursor, optionally nicotinamide riboside, and nitazoxanide.

39. The method of any one of embodiments 36-38, wherein treatment further comprises metformin.

40. The method of any one of embodiments 35-38, wherein the fatty acid liver disease is NAFLD and/or NASH.

### BRIEF DESCRIPTION OF THE DRAWINGS

In addition to the features described herein, additional features and variations will be readily apparent from the following descriptions of the drawings and exemplary embodiments. It is to be understood that these drawings depict embodiments and are not intended to be limiting in scope.

FIG. 1A depicts an embodiment of a schematic diagram of the developing steatohepatitis-human liver organoid (sHLO) with persistent insulin and fatty acid exposures under defined metabolic context. Scale bars, 200 μm.

FIG. 1B depicts an embodiment of a transcriptome analysis of HLOs from 24 donors.

FIG. 1C depicts an embodiment of representative images of sHLOs grouped based on lipid accumulation from a 24-donor HLO pool and quantification of fat accumulation by BODIPY staining, unpaired t test: ****p<0.0001.

FIG. 1D depicts an embodiment of a bar graph plotting the percentage of sHLO present from each donor in groups with high and low fat accumulation.

FIG. 1E depicts an embodiment of SNP genotype profiles associated with NAFLD in 24 donors used in the HLO panel. Dark green indicates 2 variant alleles, light green indicates 1 variant allele.

FIG. 1F depicts an embodiment of the odds ratios (ORs) for 24-donor sHLO model. The fat accumulation phenotype of HLOs and the OR of major NAFLD-related SNPs. Error bars represent 95% confidence intervals.

FIG. 1G depicts an embodiment of a comparison of diagnostic odds ratios in clinical trials and odds ratios in HLO models for PNPLA3 rs738409. Indicate the sample size (n) and minor allele frequency (MAF).

FIG. 1H depicts an embodiment of a comparison of diagnostic odds ratios in clinical trials and odds ratios in HLO models for GCKR 1260326.

FIG. 2A depicts an embodiment of a schematic diagram of GCKR variant association with glucokinase (GCK). GCKR functions as an inhibitor of GCK in the liver. The TT variant of GCKR-rs1260326 has reduced ability to bind GCK and is less effective in suppressing GCK activities.

FIG. 2B depicts an embodiment of the time-course dynamics of GCK activity in HLOs with GCKR-rs1260326 allele CC or TT. Data are shown as means±SD. (error bars), n=4.

FIG. 2C depicts an embodiment of measurement of GCK activity in HLOs carrying allele CC or TT of GCKR-rs1260326. Data are shown as mean±SD. (Error bars), n=4, triplicate. Unpaired t test; ***p<0.001.

FIG. 2D depicts an embodiment of representative images of de novo lipid accumulation in HLOs with allele CC or TT. Images were stained with BODIPY for fat accumulation (Green) and DAPI for the nucleus (Blue). Scale bars, low magnification: 300 μm, high magnification: 50 μm.

FIG. 2E depicts an embodiment of quantification of de novo lipid accumulation in HLOs with allele CC or TT. The intensity of lipid was normalized to nuclear signals (mean±SD, n=8 independent experiments). Unpaired t test; ****p<0.0001.

FIG. 2F depicts an embodiment of a comparison of lipogenesis associated gene expression in HLOs with allele CC or TT. Data are shown as means±SD. (error bars), n=4-8. Unpaired t test; *p<0.05, **p<0.01, versus CC HLO.

FIG. 2G depicts an embodiment of imaging of de novo lipid accumulation in HLOs carrying CC or TT, treated with PFKFB3 inhibitor (PFK15), and GCK-GCKR disruptor (AMG3969). Images were stained with BODIPY for fat accumulation and DAPI for the nucleus. Scale bars, low magnification: 100 μm, high magnification: 50 μm.

FIG. 2H depicts an embodiment of a quantification of de novo lipid accumulation in HLOs with allele CC or TT treated with PFK15 or AMG3969. The intensity of lipid was normalized to nuclear signals (mean±SD, n=8 independent experiments). Unpaired t test; ***p<0.001, ****p<0.0001.

FIG. 2I depicts an embodiment of an analysis of lipogenesis associated gene expression in TT-HLOs treated with PFK15 or AMG3969. Data are shown as means±SD (error bars), n=4-8. Unpaired t test; *p<0.05, **p<0.01, ****p<0.0001, versus untreated.

FIG. 3A depicts an embodiment of an impact of HbA1c values (normal, <5.7%, versus diabetic, >6.4%) on ALT measurements.

FIG. 3B depicts an embodiment of an impact of HbA1c values (normal, <5.7%, versus diabetic, >6.4%) on NAFLD activity score (NAS).

FIG. 3C depicts an embodiment of an impact of HbA1c values (normal, <5.7%, versus diabetic, >6.4%) on lobular inflammation scores.

FIG. 3D depicts an embodiment of an impact of HbA1c values (normal, <5.7%, versus diabetic, >6.4%) on SAF activity score.

FIG. 4A depicts an embodiment of a volcano plot of differentially expressed gene (DEGs) analysis (edge R) in primary NASH hepatocytes comparing GCKR TT risk to CC non-risk variants. Fold change>1.5, P-value<0.05.

FIG. 4B depicts an embodiment of an unbiased gene set enrichment analysis (GSEA). REACTOME pathways up-regulated and down-regulated in GCKR TT risk compared to CC non-risk variants in primary NASH hepatocytes. Normalized enrichment scores (NES) are presented in descending order.

FIG. 4C depicts an embodiment of conserved GSEA-REACTOME pathways in primary NASH hepatocytes (clinical samples) from b, and HLOs (GCKR-TT versus GCKR-CC). NES less than −1.6 are shown.

FIG. 4D depicts an embodiment of conserved GSEA-REACTOME mitochondrial-related pathways in clinical (primary NASH hepatocytes) and HLO models.

FIG. 4E depicts an embodiment of enrichment plots of selected gene-expression profile based on GSEA-REACTOME evaluations.

FIG. 4F depicts an embodiment of oxygen consumption rate (OCR) analysis (Extracellular Oxygen Consumption Assay, a fluorescence-based assay) of GCKR TT-HLO and -sHLO. Data are shown as means±SD. (error bars), n=3.

FIG. 4G depicts an embodiment of a ratio of ATP/AMP of CC and TT sHLO analyzed by NMR (nuclear magnetic resonance) profiles. Data are shown as means±SD. (error bars), n in CC=5, n in TT=4 donors. Unpaired t test; **p<0.01.

FIG. 4H depicts an embodiment of a quantifications of reactive oxidant species (ROS) production in sHLOs with allele CC or TT. ROS production were detected with CellROX live staining and DAPI for the nucleus. The intensity of ROS was normalized to nuclear signals. Analysis was performed in over 50 organoids per line, three independent experiments. Unpaired t test; ***p<0.001.

FIG. 5A depicts an embodiment of an oxygen consumption rate (OCR) analysis of GCKR TT-HLO, -sHLO and -sHLO with nicotinamide riboside (NR), nitazoxanide (NTZ) combination. Data are shown as means±SD. (error bars), n=3, Unpaired t test; *p<0.05, **p<0.01.

FIG. 5B depicts an embodiment of a NAD+/NADH ratios in TT sHLO treated with NR, NTZ, or combination.

FIG. 5C depicts an embodiment of representative images of ROS production in TT-HLO, -sHLO (FFA treated) and -sHLO untreated or treated with metformin (MET) or a combination of NR/NTZ. Images were stained with CellROX for ROS, and DAPI for the nucleus. Scale bars, 300 μm.

FIG. 5D depicts an embodiment of quantifications of ROS production in TT-HLO, -sHLO (FFA treated) and -sHLO untreated or treated with metformin (MET) or a combination of NR/NTZ. ROS production were detected with CellROX live staining and DAPI for the nucleus. The intensity of ROS was normalized to nuclear signals. Analysis was performed in over 50 organoids per line, three independent experiments. Unpaired t test; ****p<0.0001.

FIG. 5E depicts an embodiment of relative gene expressions of proinflammatory cytokine in TT-sHLO (FFA treated) and -sHLO untreated or treated with metformin (MET) or NR/NTZ combination were compared to TT-HLO, which was arbitrarily assigned a value of 1. Data are shown as means±SD. (error bars), n=4. Unpaired t test; *p<0.05, **p<0.01.

FIG. 6 depicts an embodiment of a dataset that included 1089 adults from the STELLAR-3 (NCT03053050) and ATLAS (NCT03449446) trials who were diagnosed with NAFLD and characterized demographics, biomarkers, and liver histology of clinical samples. Demographics were predominantly caucasian, middle-aged, females with high BMI in obese ranges. Hemoglobin A1C values were not available for only two samples out of a total of 1091 sample information. P values were analyzed for reference allele and alternate allele.

FIG. 7A depicts an embodiment of a schematic diagram of the developing human liver organoid panel from multiple donor-derived foregut mixed progenitors.

FIG. 7B depicts an embodiment of an optimization of organoid formation medium from frozen foregut (FG) cells. HLO formation was performed under culture conditions of 10 μM Rock inhibitor, 2 μM retinoid acid, 5 ng/mL fibroblast growth factor 2 (FGF2), 3 μM CHIR99021 (GSK-3 inhibitor), 0.5 μM A83-01 (TGFβ inhibitor) and combination. Top row: bright field image of the HLOs in the Matrigel drop. Bottom row: live cell fluorescence staining image of HLO. Live HLOs were stained green with Calcein-AM. Scale bar is 500 μm.

FIG. 7C depicts an embodiment of a quantification of the number and perimeter of HLOs formed under different culture conditions. The combination of FGF2, TGFb inhibitor and GSK3 inhibitor was the most efficient condition for HLO formation and HLO growth. The HLOs extracted from Matrigel were quantified using KEYENCE analyze software (mean±SD, n=3 independent experiments). Unpaired t-test; *p<0.05, **p<0.01, ***p<0.001.

FIG. 7D depicts an embodiment of a confirmation of clonality of HLOs formed under optimized culture condition. HLO formation was performed under mixed conditions of GFP labeled (green fluorescent protein)-FG cells and mCherry labeled (red fluorescent protein)-FG cells derived from different donors to confirm clonality. Time-lapsed series of images of HLO formation from a single FG cell with GFP or mCherry are shown. HLOs with GFP and mCherry fluorescence were formed on day 10 of culture. Scale bar is 500 μm.

FIG. 7E depicts an embodiment of a quantification of HLOs formed under mixed conditions of GFP and mCherry-FG. The HLOs were quantified using KEYENCE analyze software (mean±SD, n=8 independent experiments).

FIG. 7F-G depicts an embodiment of large scale HLO panel formation using FGs from 20 donors. HLO panels were formed from 20 donor FG cells under optimized culture conditions and the presence of 20 donors was confirmed. Donors were identified by extracting gDNA from each HLO and genotyping with donor-specific SNP recognition probes. The heat map shows that all 20 donors are included in the HLO panel, and the brightfield image shows that the morphologies of the HLOs are comparable.

FIG. 7H depicts an embodiment of a confirmation of clonality of HLOs in a large HLO panel formed from FGs of 20 donors. The percentage of chimeric HLOs detected when gDNA of each HLO was extracted and donor identification was performed by specific SNP genotyping. HLOs with two donors detected accounted for 6.2% of all HLOs and those with three or more donors accounted for 1.65% of all HLOs. A total of 384 HLOs were picked up from a 20-donor HLO panel and gDNA was extracted for genotyping.

FIG. 8A depicts an embodiment of a schematic diagram of the Donor decoding method using donor-specific SNP genotyping of HLO panels. The gDNA of each donor-derived iPSC was extracted and the SNP profile was obtained by SNP array. Based on the SNP profile, donor-specific SNP profiles were constructed. Standard (STD) curves for each donor were generated using donor gDNA mixed in arbitrary ratios. The gDNA of the multi-donor HLO panel was extracted in batches and the ratio of each donor was determined using the STD curve.

FIG. 8B depicts an embodiment of a STD curve for donor ratio quantification was prepared by mixing gDNA in an arbitrary ratio. STD curves of three representative donors are shown. R-squared values of 0.99 or higher for all STD curves were used.

FIG. 8C-D depicts an embodiment of a donor ratio quantification by SNP-STD curve. To confirm the accuracy of the donor quantification STD curve for specific SNPs, samples containing a mixture of GFP-HLO and mCherry-HLO in arbitrary proportions were performed. c. fluorescence images of HLOs mixed in proportions indicated. Donor mixing ratios were 1:99, 10:90 and 50:50. d. specific SNP PCR of the gDNA of the mixed HLOs extracted in batches to obtain the ACT value, with the mixing ratio calculated using the STD curve. The left figure shows a plot of the actual mixing ratio and the mixing ratio calculated from the STD curve. R-squared value was 0.9990. The right figure shows the ratio of GFP-HLO and mCherry-HLO from samples mixed in arbitrary ratios, calculated using the SNP-STD curve.

FIG. 9A-B depicts an embodiment of live imaging of accumulating lipid droplets in fatty acid loading HLO. Shown, HLO without fatty acid loading and sHLO treated with fatty acid for 72 hours. Lipid droplets stained green with BODIPY and nuclei are stained blue with Hoechst 33342. Scale bars, 50 μm.

FIG. 9C-D depicts an embodiment of imaging of lipid droplets by transmission electron microscopy (TEM). c represents HLO and d represents sHLO. Yellow arrows indicate accumulated lipid droplets. Scale bars, 10 μm.

FIG. 9E depicts an embodiment of an analysis of non-polar metabolites of sHLO by nuclear magnetic resonance (NMR) spectroscopy. Metabolites in the culture supernatant of sHLO induced by fatty acid treatment for 72 h were measured. Quantifications were performed in five independent hPSC lines, unpaired t test: *p<0.05, ****p<0.0001.

FIG. 9F depicts an embodiment of an ELISA analysis of proinflammatory cytokine secretion in sHLO. Proinflammatory cytokines were measured in the culture supernatant of sHLO induced by 72 hours fatty acid treatment. Data are shown as means±SD. (error bars), n=9. Unpaired t test; *p<0.05, **p<0.01.

FIG. 9G depicts an embodiment of an analysis of proinflammatory cytokine gene expressions in sHLO. Proinflammatory cytokine gene expression was measured by qPCR in sHLOs induced by 72 hours of fatty acid treatment. Data are shown as means±SD. (error bars), n=4.

FIG. 10A depicts an embodiment of a measurement of glucose production in HLO and sHLO. The amount of glucose was normalized to the CellTiter-Glo value (CTGX10\hat{ }6, mean±SD, n=3 independent experiments per line). Unpaired t test; ***p<0.001.

FIG. 10B depicts an embodiment of a gene expression analysis (qPCR) of PCK1 in HLO and sHLO. (mean±SD, n=8 independent experiments). Unpaired t test; ***p<0.001

FIG. 10C-D depicts an embodiment of a western blot analysis to determine insulin responsiveness of sHLO. HLOs and sHLOs were starved for 24 hours and treated with 1 μg/ml of insulin for 20 minutes. Density of phosphorylated AKT(T308) plotted normalized by AKT. (mean±SD, n=4 independent experiments). Unpaired t test; **p<0.01.

FIG. 10E depicts an embodiment of an insulin mediated inhibition of glucose production in sHLO. HLOs and sHLOs were incubated with insulin and serum starvation for 24 hours and treated with 1 μg/ml of insulin in a glucose-free medium. After 18 hours, the supernatant was collected and measured for glucose. The amount of glucose was normalized to CTG (mean±SD, n=3 independent experiments). Unpaired t test; ***p<0.001.

FIG. 10F depicts an embodiment of an expression of insulin-regulated glycogenesis genes in sHLO. HLO and sHLO were incubated serum starvation for 24 hours. Total RNA was collected and qPCR analyzed 8 hours after treatment with 1 μg/ml insulin. Data are shown as means±SD. (error bars), n=4. Unpaired t test; *p<0.05, **p<0.01, ***p<0.001, ****p<0.0001 versus untreated.

FIG. 11A depicts an embodiment of a graphic depiction of selected ALT measurements. from Table 1, delineated by PNPLA3 variant and HbA1c values.

FIG. 11B depicts an embodiment of a graphic depiction of selected NAFLD activity score (NAS) from Table 1, delineated by PNPLA3 variant and HbA1c values.

FIG. 11C depicts an embodiment of a graphic depiction of selected lobular inflammation scores from Table 1, delineated by PNPLA3 variant and HbA1c values.

FIG. 11D depicts an embodiment of a graphic depiction of selected SAF activity scores from Table 1, delineated by PNPLA3 variant and HbA1c values.

FIG. 12A depicts an embodiment of live imaging of accumulating lipid droplets in sHLO with GCKR CC or TT alleles. Images were stained with BODIPY for fat accumulation (Green) and DAPI for the nucleus (Blue).

FIG. 12B depicts an embodiment of quantification of lipid droplet in sHLOs. The intensity of lipid was normalized to nuclear signals. Unpaired t test; ****p<0.0001.

FIG. 12C depicts an embodiment of a qPCR analysis of proinflammatory cytokine gene expressions in sHLO. Data are shown as means±SD. (error bars), n=4.

FIG. 12D depicts an embodiment of pathways enriched in GCKR-TT HLOs, compared to GCKR-CC HLOs, with NES>1.2.

FIG. 12E depicts an embodiment of a GSEA evaluation of gene-expression profile enriched in GCKR-TT sHLO. REACTOME FATTY ACIDS (NES 1.68, p-value=0.002, FDR<0.3039), REACTOME INTERFERON GAMMA SIGNALING (NES 1.77, p-value<0.001 FDR<0.068).

FIG. 13 depicts an embodiment of a graphic depiction of the effects of metformin treatment on NAS. Patients carrying GCKR TT did not show improvement in NAS after 48 weeks of metformin treatment, in contrast to patients carrying GCKR CC or CT.

FIG. 14 depicts an embodiment of iPSC line information.

FIG. 15 depicts an embodiment of demographic and baseline characteristics in a cohort of 1091 NAFLD patients

FIG. 16 depicts an embodiment of clinical sample information provided in the RNA sequence.

FIG. 17 depicts an embodiment of liver tests and histology after 48 weeks of metformin treatment in NAFLD patients.

FIG. 18 depicts an embodiment of a list of primers sets.

### DETAILED DESCRIPTION

Since NAFLD and T2D are often present in the same patients, a full understanding of the pleiotropic roles of candidate variants, including glucokinase regulatory protein (GCKR) rs1260326 SNP (single nucleotide polymorphism), is essential for providing more insightful diagnosis and prognosis.

The use of in-a-dish organ systems are being explored as models that allow genotype and phenotype association studies under a defined metabolic context in the absence of other major confounding factors. Recent genome-wide association studies, GWAS-in-a-dish efforts, using metabolic cell types differentiated from induced pluripotent stem cells (iPSCs), have validated human gene expression variation such as gene expression quantitative trait loci (eQTL). Emerging organoid-based approaches can further increase the potential of this in-a-dish strategy as organoids emulate anatomical and physiological characteristics of in vivo organs in health and disease.

Herein, a pooled human organoid-based strategy for steatohepatitis genotype-phenotype association studies is utilized. Steatohepatitis-like organoids from multiple genotyped individuals can be simultaneously phenotyped under the same culture conditions, enabling efficient evaluation of genetic association for NAFLD. Organoid informed genetic and molecular mechanisms were integrated with available extensive clinical data obtained during the course of three randomized controlled trials of therapies for patients with advanced fibrosis due to NASH. It is demonstrated that the highly controversial GCKR rs1260326 variant is clinically significant for NAFLD and T2D based on results from the organoid models showing that the functional significance of the GCKR rs1260326 variant is dependent on the metabolic status, influenced by the inflammatory milieu. Metabolically-resolved genetic and phenotypic assessments will be critical to identify biomarkers and tailor interventional strategies.

‘GWAS in-a-dish’ is a strategy to determine the personalized phenotypes in a collection of cells from multiple individuals. A GWAS in-a-dish concept was integrated with an organoid-based functional approach to capture pathological genetic variations associated with NAFLD/NASH. This in vitro manipulatable approach for evaluating heritable variants circumvented the numerous in vivo non-heritable confounders, including lifestyle and nutrition, which perturb NAFLD/NASH and other metabolism dependent diseases, and which often lead to controversial interpretation of discovered variants. The strategy of pooling iPSCs-derived foregut progenitors, moreover, provide an alternative to conventional laboratory scale protocols which are generally ill equipped to perform large-scale phenotypic analyses as the cost is prohibitive and procedures are labor intensive. The improved differentiation methodologies led to successful parallel and clonal differentiation of the pooled foregut progenitors into HLOs. The clonal HLOs were conducive to en masse screening for quantifying donor-specific intra-hepatocytic lipid levels, an early pathophysiological manifestation of NAFLD. It was found that the scaling of lipid accumulation was strikingly influenced by known NAFLD risk variants. Hence, this pooled iPSC-derived foregut progenitors enabled: 1) application of identical pathologic insults; 2) live tracking and sorting of organoids utilizing fluorescent readouts; and 3) SNP profiling associated with the organoid-of-origin encompassing phenotypic information. As a proof-of-principle, the pooled HLO genotype-phenotype association studies informed the impact of key, GWAS identified, NAFLD risk alleles on liver steatosis phenotype. Thus, this pooling strategy represents a first-of-a-kind organoid level ‘forward cellomics’ platform to interrogate genotype-driven phenotypic association in human organoid models.

The organoid models are viable human-based systems for evaluating in-depth phenotypic impact of an identified variant, independent of patient metabolic status. It was demonstrated that the clinically controversial GCKR-rs1260326 TT risk variant, in pooled and individually assessed HLOs, was biologically significant under culturing conditions which mimic T2D insulin resistance. In addition to enhanced fatty-acid-induced TG accumulation with correlating inflammatory signatures, DNL, insulin resistance, and mitochondrial dysfunction were demonstrable and distinguishable from HLOs carrying non-risk GCKR variants. These differential functional evidence of metabolic perturbations in HLOs provided unique insights to the contribution of the GCKR risk variant in NALFD and is indicative of the vast potential of human organoids for mechanistic studies.

In vivo, the prognostic value of a T2D phenotype for patients carrying GCKR-rs1260326 TT was highlighted by the discovery that HbA1c measurements, a diagnostic indicator for T2D, uniquely delineated the severity of NAFLD/NASH-associated inflammatory pathologies. Patients with T2D diabetic HbA1c values (>6.4%) were associated with more severe pathologies than those with normal HbA1c values (<5.7%). This differentiation factor was not observed for other genetic risk variants, particularly the well-established PNPLA3 rs728409 GG risk variant. Inclusion of HbA1c measurements, which are often missing, is important in clinical studies of GCKR-rs1260326 cohorts. The influence of ethnicity, gender, age, and BMI status on differential HbA1c values in GCKR-rs1260326 cohorts, remains to be determined.

Since T2D complication in NAFLD patients present with hepatic insulin resistance, the GCKR rs1260326 TT dependent subgrouping by HbA1c values are informative for precision patient management strategies. The subgroup of patients who had non-diabetic HbA1c values (<5.7%), exhibited improved pathologies upon metformin therapy while patients with T2D indications (HbA1c>6.4%), and the T2D-like HLO models, were poorly responsive to metformin. The implication, for risk carriers, is the possibility that reduction of dietary fat supplementation may alleviate hepatic substrate dependent lipogenesis, improving insulin resistant state and suppressing lipid deposition. Such non-medical treatments, i.e. lifestyle modification and weight loss, which continues to be recommended as alternatives to medication despite high variability in outcomes, could be beneficial when integrated with understanding the genotype-driven physiological condition of the patient. The contribution of GCKR-rs1260326 TT risk variant to NAFLD/NASH is highly dependent on the diabetic status informed by HbA1c measurements, and, thus, is of prognostic value for GCKR-rs1260326 TT risk carriers. The results also pave the way for designing focused approaches to better address the T2D complication in populations of GCKR risk carriers, by optimally controlling substrate intake and de novo lipogenesis via lifestyle modification and/or drug exposure. Collectively, the integration of in vitro HLO models with in vivo clinical data provided new insights to improve capturing the highly variable in vivo pathogenesis of NAFLD/NASH risk variants.

GCKR is almost exclusively expressed in the liver. Evidence in support of the role of GCKR in liver diseases include a rare loss-of-function GCKR variant Arg227Ter which is associated with a rapidly progressive form of nonalcoholic steatohepatitis. The GCKR rs1260326 TT polymorphism (resulting in the missense Pro446Leu), in contrast, is commonly found in non-African population and has been recognized for its critical role in fatty liver-associated hepatic insulin resistance. However, the missense GCKR p.Pro446Leu, with loss of ability to interact and modulate GCK activities, is also recognized to facilitate and enhance hepatic glucose utilization and strongly implicated in lower fasting glucose, thereby bestowing protection against T2D. These observations, albeit contradictory, had led to development of biologics disrupting GCK-GCKR complex interactions, but initial therapeutic promise has been hampered by undesirable side-effects of hypoglycemia, increased hepatic steatosis (as we also observed in our HLO model), and loss of efficacy presumably related to the development of hepatic insulin resistance. The data are consistent with the so-called “double-edged sword” for GCKR actions, in which the contribution of GCKR to NAFLD was suggested to be both protective and pathogenic. Based on the integrated HLO and clinical findings, that the mechanistic consequence of GCKR Pro446Leu favor hepatic fat accumulation by activation of GCK, and the contradictory roles of GCKR can be parsed, depending on the metabolic, genetic and diabetic status of the patient.

It was discovered that the GCKR-rs1260326 TT risk variant (Pro446Leu) was strongly associated with mitochondrial dysregulation, an association not previously reported. This was evidenced from transcriptomic analyses of clinical samples (hepatocytes) and HLO models and supported by functional demonstrations of potently enhanced persistent mitochondrial ROS and reduced OCR, which were exacerbated by exposure to fatty acid. Intriguingly, treating our TT HLO steatohepatitis-like models with oxidative uncouplers (NR/NTZ) but not with metformin, normalized mitochondrial functions and suppressed fatty-acid induced inflammatory response, providing new therapeutic options.

Emerging therapeutic approaches to treat NAFLD and T2D have focused on disrupting metabolic and inflammatory pathways that interconnect the two conditions. Efficacy, however, has remained poor, limited or controversial, due, in part, to still imperfect understanding of these complex interconnections in humans, and the roles and contributions of genomic SNPs. For carriers of the GCKR rs1260326 variant TT (patients and HLO models), metformin, and developing NASH drugs obeticholic acid and CCR2/5 inhibitor (not shown), demonstrated poor pharmacological benefits in suppressing NASH-associated inflammatory phenotypes. Unexpected findings that an FDA-approved drug, NTZ, in concert with NR, was capable of suppressing lipid-induced inflammation in HLO carrying GCKR rs1260326 TT, support effective pharmacological intervention for at least a subset of patients with NAFLD. Since the GCKR rs1260326 TT risk variant represents almost 40% of the US population, with African ethnicity having the lowest (app. 10%) allelic frequency, there is a better understanding of the mechanism in which risk variants contribute to NAFLD and new targets for therapeutic intervention. The importance of delineating key predispositions will allow more accurate identification of patients who are in need of primary and/or secondary prevention.

Integrating organoid modeling and clinical analyses highlights methodological advances and emphasize new insights for better understanding of the personalized basis of complex, common, diseases such as NAFLD/NASH. Improvement in patient stratification will enable earlier identification and implementation of preventive and therapeutic strategies. With NAFLD affecting nearly one billion people globally, early identification of susceptible individuals with a rigorous interventional design including lifestyle management, and refining treatment options, is critical.

### Terms

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Unless defined otherwise, technical and scientific terms used herein have the same meaning as commonly understood when read in light of the instant disclosure by one of ordinary skill in the art to which the present disclosure belongs. For purposes of the present disclosure, the following terms are explained below.

The disclosure herein uses affirmative language to describe the numerous embodiments. The disclosure also includes embodiments in which subject matter is excluded, in full or in part, such as substances or materials, method steps and conditions, protocols, or procedures.

The articles “a” and “an” are used herein to refer to one or to more than one (for example, at least one) of the grammatical object of the article. By way of example, “an element” means one element or more than one element.

By “about” is meant a quantity, level, value, number, frequency, percentage, dimension, size, amount, weight or length that varies by as much as 10% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The terms “individual”, “subject”, or “patient” as used herein have their plain and ordinary meaning as understood in light of the specification, and mean a human or a non-human mammal, e.g., a dog, a cat, a mouse, a rat, a cow, a sheep, a pig, a goat, a non-human primate, or a bird, e.g., a chicken, as well as any other vertebrate or invertebrate. The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea pigs, or the like.

The terms “effective amount” or “effective dose” as used herein have their plain and ordinary meaning as understood in light of the specification, and refer to that amount of a recited composition or compound that results in an observable effect. Actual dosage levels of active ingredients in an active composition of the presently disclosed subject matter can be varied so as to administer an amount of the active composition or compound that is effective to achieve the desired response for a particular subject and/or application. The selected dosage level will depend upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

The terms “function” and “functional” as used herein have their plain and ordinary meaning as understood in light of the specification, and refer to a biological, enzymatic, or therapeutic function.

The term “inhibit” as used herein has its plain and ordinary meaning as understood in light of the specification, and may refer to the reduction or prevention of a biological activity. The reduction can be by a percentage that is, is about, is at least, is at least about, is not more than, or is not more than about, 10%, 20%, 30%, 40%, 50%, 60%, 70%, 80%, 90%, or 100%, or an amount that is within a range defined by any two of the aforementioned values. As used herein, the term “delay” has its plain and ordinary meaning as understood in light of the specification, and refers to a slowing, postponement, or deferment of a biological event, to a time which is later than would otherwise be expected. The delay can be a delay of a percentage that is, is about, is at least, is at least about, is not more than, or is not more than about, 0%, 10%, 20%, 30%, 40%, 50%, 60%, 70%, 80%, 90%, 100%, or an amount within a range defined by any two of the aforementioned values. The terms inhibit and delay may not necessarily indicate a 100% inhibition or delay. A partial inhibition or delay may be realized.

As used herein, the term “isolated” has its plain and ordinary meaning as understood in light of the specification, and refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from equal to, about, at least, at least about, not more than, or not more than about, 10%, about 20%, about 30%, about 40%, about 50%, about 60%, about 70%, about 80%, about 90%, about 95%, about 98%, about 99%, substantially 100%, or 100% of the other components with which they were initially associated (or ranges including and/or spanning the aforementioned values). In some embodiments, isolated agents are, are about, are at least, are at least about, are not more than, or are not more than about 80%, about 85%, about 90%, about 91%, about 92%, about 93%, about 94%, about 95%, about 96%, about 97%, about 98%, about 99%, substantially 100%, or 100% pure (or ranges including and/or spanning the aforementioned values). As used herein, a substance that is “isolated” may be “pure” (e.g., substantially free of other components). As used herein, the term “isolated cell” may refer to a cell not contained in a multi-cellular organism or tissue.

As used herein, “in vivo” is given its plain and ordinary meaning as understood in light of the specification and refers to the performance of a method inside living organisms, usually animals, mammals, including humans, and plants, as opposed to a tissue extract or dead organism.

As used herein, “ex vivo” is given its plain and ordinary meaning as understood in light of the specification and refers to the performance of a method outside a living organism with little alteration of natural conditions.

As used herein, “in vitro” is given its plain and ordinary meaning as understood in light of the specification and refers to the performance of a method outside of biological conditions, e.g., in a petri dish or test tube.

The terms “nucleic acid” or “nucleic acid molecule” as used herein have their plain and ordinary meaning as understood in light of the specification, and refer to polynucleotides, such as deoxyribonucleic acid (DNA) or ribonucleic acid (RNA), oligonucleotides, those that appear in a cell naturally, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate. The term “nucleic acid molecule” also includes so-called “peptide nucleic acids,” which comprise naturally-occurring or modified nucleic acid bases attached to a polyamide backbone. Nucleic acids can be either single stranded or double stranded. “Oligonucleotide” can be used interchangeable with nucleic acid and can refer to either double stranded or single stranded DNA or RNA. A nucleic acid or nucleic acids can be contained in a nucleic acid vector or nucleic acid construct (e.g. plasmid, virus, retrovirus, lentivirus, bacteriophage, cosmid, fosmid, phagemid, bacterial artificial chromosome (BAC), yeast artificial chromosome (YAC), or human artificial chromosome (HAC)) that can be used for amplification and/or expression of the nucleic acid or nucleic acids in various biological systems. Typically, the vector or construct will also contain elements including but not limited to promoters, enhancers, terminators, inducers, ribosome binding sites, translation initiation sites, start codons, stop codons, polyadenylation signals, origins of replication, cloning sites, multiple cloning sites, restriction enzyme sites, epitopes, reporter genes, selection markers, antibiotic selection markers, targeting sequences, peptide purification tags, or accessory genes, or any combination thereof.

A nucleic acid or nucleic acid molecule can comprise one or more sequences encoding different peptides, polypeptides, or proteins. These one or more sequences can be joined in the same nucleic acid or nucleic acid molecule adjacently, or with extra nucleic acids in between, e.g. linkers, repeats or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a nucleic acid as used herein has its plain and ordinary meaning as understood in light of the specification and refers to a sequence being after the 3′-end of a previous sequence, on the strand containing the encoding sequence (sense strand) if the nucleic acid is double stranded. The term “upstream” on a nucleic acid as used herein has its plain and ordinary meaning as understood in light of the specification and refers to a sequence being before the 5′-end of a subsequent sequence, on the strand containing the encoding sequence (sense strand) if the nucleic acid is double stranded. The term “grouped” on a nucleic acid as used herein has its plain and ordinary meaning as understood in light of the specification and refers to two or more sequences that occur in proximity either directly or with extra nucleic acids in between, e.g. linkers, repeats, or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths, but generally not with a sequence in between that encodes for a functioning or catalytic polypeptide, protein, or protein domain.

The nucleic acids described herein comprise nucleobases. Primary, canonical, natural, or unmodified bases are adenine, cytosine, guanine, thymine, and uracil. Other nucleobases include but are not limited to purines, pyrimidines, modified nucleobases, 5-methylcytosine, pseudouridine, dihydrouridine, inosine, 7-methylguanosine, hypoxanthine, xanthine, 5,6-dihydrouracil, 5-hydroxymethylcytosine, 5-bromouracil, isoguanine, isocytosine, aminoallyl bases, dye-labeled bases, fluorescent bases, or biotin-labeled bases.

The terms “peptide”, “polypeptide”, and “protein” as used herein have their plain and ordinary meaning as understood in light of the specification and refer to macromolecules comprised of amino acids linked by peptide bonds. The numerous functions of peptides, polypeptides, and proteins are known in the art, and include but are not limited to enzymes, structure, transport, defense, hormones, or signaling. Peptides, polypeptides, and proteins are often, but not always, produced biologically by a ribosomal complex using a nucleic acid template, although chemical syntheses are also available. By manipulating the nucleic acid template, peptide, polypeptide, and protein mutations such as substitutions, deletions, truncations, additions, duplications, or fusions of more than one peptide, polypeptide, or protein can be performed. These fusions of more than one peptide, polypeptide, or protein can be joined in the same molecule adjacently, or with extra amino acids in between, e.g. linkers, repeats, epitopes, or tags, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a polypeptide as used herein has its plain and ordinary meaning as understood in light of the specification and refers to a sequence being after the C-terminus of a previous sequence. The term “upstream” on a polypeptide as used herein has its plain and ordinary meaning as understood in light of the specification and refers to a sequence being before the N-terminus of a subsequent sequence.

The term “purity” of any given substance, compound, or material as used herein has its plain and ordinary meaning as understood in light of the specification and refers to the actual abundance of the substance, compound, or material relative to the expected abundance. For example, the substance, compound, or material may be at least 80, 85, 90, 91, 92, 93, 94, 95, 96, 97, 98, 99, or 100% pure, including all decimals in between. Purity can be affected by unwanted impurities, including but not limited to nucleic acids, DNA, RNA, nucleotides, proteins, polypeptides, peptides, amino acids, lipids, cell membrane, cell debris, small molecules, degradation products, solvent, carrier, vehicle, or contaminants, or any combination thereof. In some embodiments, the substance, compound, or material is substantially free of host cell proteins, host cell nucleic acids, plasmid DNA, contaminating viruses, proteasomes, host cell culture components, process related components, mycoplasma, pyrogens, bacterial endotoxins, and adventitious agents. Purity can be measured using technologies including but not limited to electrophoresis, SDS-PAGE, capillary electrophoresis, PCR, rtPCR, qPCR, chromatography, liquid chromatography, gas chromatography, thin layer chromatography, enzyme-linked immunosorbent assay (ELISA), spectroscopy, UV-visible spectrometry, infrared spectrometry, mass spectrometry, nuclear magnetic resonance, gravimetry, or titration, or any combination thereof.

The term “yield” of any given substance, compound, or material as used herein has its plain and ordinary meaning as understood in light of the specification and refers to the actual overall amount of the substance, compound, or material relative to the expected overall amount. For example, the yield of the substance, compound, or material is, is about, is at least, is at least about, is not more than, or is not more than about, 80, 85, 90, 91, 92, 93, 94, 95, 96, 97, 98, 99, or 100% of the expected overall amount, including all decimals in between. Yield can be affected by the efficiency of a reaction or process, unwanted side reactions, degradation, quality of the input substances, compounds, or materials, or loss of the desired substance, compound, or material during any step of the production.

As used herein, “pharmaceutically acceptable” has its plain and ordinary meaning as understood in light of the specification and refers to carriers, excipients, and/or stabilizers that are nontoxic to the cell or mammal being exposed thereto at the dosages and concentrations employed or that have an acceptable level of toxicity. A “pharmaceutically acceptable” “diluent,” “excipient,” and/or “carrier” as used herein have their plain and ordinary meaning as understood in light of the specification and are intended to include any and all solvents, dispersion media, coatings, antibacterial and antifungal agents, isotonic and absorption delaying agents, and the like, compatible with administration to humans, cats, dogs, or other vertebrate hosts. Typically, a pharmaceutically acceptable diluent, excipient, and/or carrier is a diluent, excipient, and/or carrier approved by a regulatory agency of a Federal, a state government, or other regulatory agency, or listed in the U.S. Pharmacopeia or other generally recognized pharmacopeia for use in animals, including humans as well as non-human mammals, such as cats and dogs. The term diluent, excipient, and/or “carrier” can refer to a diluent, adjuvant, excipient, or vehicle with which the pharmaceutical composition is administered. Such pharmaceutical diluent, excipient, and/or carriers can be sterile liquids, such as water and oils, including those of petroleum, animal, vegetable or synthetic origin. Water, saline solutions and aqueous dextrose and glycerol solutions can be employed as liquid diluents, excipients, and/or carriers, particularly for injectable solutions. Suitable pharmaceutical diluents and/or excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. A non-limiting example of a physiologically acceptable carrier is an aqueous pH buffered solution. The physiologically acceptable carrier may also comprise one or more of the following: antioxidants, such as ascorbic acid, low molecular weight (less than about 10 residues) polypeptides, proteins, such as serum albumin, gelatin, immunoglobulins, hydrophilic polymers such as polyvinylpyrrolidone, amino acids, carbohydrates such as glucose, mannose, or dextrins, chelating agents such as EDTA, sugar alcohols such as mannitol or sorbitol, salt-forming counterions such as sodium, and nonionic surfactants such as TWEEN®, polyethylene glycol (PEG), and PLURONICS®. The composition, if desired, can also contain minor amounts of wetting, bulking, emulsifying agents, or pH buffering agents. These compositions can take the form of solutions, suspensions, emulsion, sustained release formulations and the like. The formulation should suit the mode of administration.

Cryoprotectants are cell composition additives to improve efficiency and yield of low temperature cryopreservation by preventing formation of large ice crystals. Cryoprotectants include but are not limited to DMSO, ethylene glycol, glycerol, propylene glycol, trehalose, formamide, methyl-formamide, dimethyl-formamide, glycerol 3-phosphate, proline, sorbitol, diethyl glycol, sucrose, triethylene glycol, polyvinyl alcohol, polyethylene glycol, or hydroxyethyl starch. Cryoprotectants can be used as part of a cryopreservation medium, which include other components such as nutrients (e.g. albumin, serum, bovine serum, fetal calf serum [FCS]) to enhance post-thawing survivability of the cells. In these cryopreservation media, at least one cryoprotectant may be found at a concentration that is, is about, is at least, is at least about, is not more than, or is not more than about, 0.01%, 0.05%, 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90%, or any percentage within a range defined by any two of the aforementioned numbers.

Additional excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), citric acid, salts, sodium chloride, sodium bicarbonate, sodium phosphate, sodium borate, sodium citrate, potassium chloride, potassium phosphate, magnesium sulfate sugars, dextrose, fructose, mannose, lactose, galactose, sucrose, sorbitol, cellulose, serum, amino acids, polysorbate 20, polysorbate 80, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. Some excipients may be in residual amounts or contaminants from the process of manufacturing, including but not limited to serum, albumin, ovalbumin, antibiotics, inactivating agents, formaldehyde, glutaraldehyde, β-propiolactone, gelatin, cell debris, nucleic acids, peptides, amino acids, or growth medium components or any combination thereof. The amount of the excipient may be found in composition at a percentage that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

The term “pharmaceutically acceptable salts” has its plain and ordinary meaning as understood in light of the specification and includes relatively non-toxic, inorganic and organic acid, or base addition salts of compositions or excipients, including without limitation, analgesic agents, therapeutic agents, other materials, and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc, and the like. Salts may also be formed with suitable organic bases, including those that are non-toxic and strong enough to form such salts. For example, the class of such organic bases may include but are not limited to mono-, di-, and trialkylamines, including methylamine, dimethylamine, and triethylamine; mono-, di-, or trihydroxyalkylamines including mono-, di-, and triethanolamine; amino acids, including glycine, arginine and lysine; guanidine; N-methylglucosamine; N-methylglucamine; L-glutamine; N-methylpiperazine; morpholine; ethylenediamine; N-benzylphenethylamine; trihydroxymethyl aminoethane.

Proper formulation is dependent upon the route of administration chosen. Techniques for formulation and administration of the compounds described herein are known to those skilled in the art. Multiple techniques of administering a compound exist in the art including, but not limited to, enteral, oral, rectal, topical, sublingual, buccal, intraaural, epidural, epicutaneous, aerosol, parenteral delivery, including intramuscular, subcutaneous, intra-arterial, intravenous, intraportal, intra-articular, intradermal, peritoneal, intramedullary injections, intrathecal, direct intraventricular, intraperitoneal, intranasal or intraocular injections. Pharmaceutical compositions will generally be tailored to the specific intended route of administration.

As used herein, a “carrier” has its plain and ordinary meaning as understood in light of the specification and refers to a compound, particle, solid, semi-solid, liquid, or diluent that facilitates the passage, delivery and/or incorporation of a compound to cells, tissues and/or bodily organs.

As used herein, a “diluent” has its plain and ordinary meaning as understood in light of the specification and refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

The term “basement membrane matrix” or “extracellular matrix” as used herein has its plain and ordinary meaning in light of the specification and refers to any biological or synthetic compound, substance, or composition that enhances cell attachment and/or growth. Any extracellular matrix, as well as any mimetic or derivative thereof, known in the art can be used for the methods disclosed herein. Some examples of extracellular matrices, or mimetics or derivative thereof, include but are not limited to cell-based feeder layers, polymers, proteins, polypeptides, nucleic acids, sugars, lipids, poly-lysine, poly-ornithine, collagen, collagen IV, gelatin, fibronectin, vitronectin, laminin, laminin-511 elastin, tenascin, heparan sulfate, entactin, nidogen, osteopontin, perlecan, basement membrane, Matrigel, hydrogel, PEI, WGA, or hyaluronic acid, or any combination thereof. A common basement membrane matrix that is used in laboratories are those isolated from murine Engelbreth-Holm-Swarm (EHS) sarcoma cells. However, these basement membrane matrices are derived from non-human animals and therefore contain xenogeneic components that prevent its use towards humans. They are also not defined, which can lead to variability in manufacturing, as well as potentially harbor pathogens. Accordingly, in some embodiments, the methods for culturing cells may involve the use of synthetic and/or defined alternatives to these xenogeneic basement membrane matrices. The use of non-xenogeneic basement membrane matrices or mimetics or derivatives thereof enables manufacturing of biological products better suited for human use.

The terms “passage” and “passaging” as used herein have their plain and ordinary meaning as understood in light of the specification, and refer to the conventional approaches performed in biological cell culture methods to maintain a viable population of cells for prolonged periods of time. As cells are generally proliferative in cell culture, they undergo multiple cycles of mitosis until occupying the available space, which is typically a surface of a cell culture container (e.g., a plate, dish, or flask) submerged under culture medium. For example, the cells may grow out as a monolayer on a cell culture container surface. If the growing cells occupy the entire available space of surface, they cannot proliferate further and may exhibit senescent behavior. In order to continue growth of the cells, which may be performed to maintain the viability and proliferative nature of the cells and/or to expand the number of cells for downstream purposes, the cells may be passaged by taking a fraction of the cells and seeding this fraction onto a fresh surface (e.g., of a cell culture container) in culture medium. This fraction of the cells will continue to proliferate and multiply until they occupy the available space of the new surface, upon which this passaging can be repeated successively.

The microscopic architecture of the liver is made up of polygonal structures called “hepatic lobules”. Classically, these lobules take on a hexagonal structure, although other geometric shapes are observed depending on tissue specification. Each lobule unit comprises plates or layers of hepatocytes surrounding an internal central vein and encapsulated by bundles of vessels called portal triads, which are made up of a portal vein, hepatic artery, and bile duct. Hepatic activity occurs as blood flows from the portal triads at the periphery, across the hepatocytes, and into the central vein to return to the circulatory system. Due to the asymmetric organization of these lobules, the layers of hepatocytes are divided into three zones. Cells in the “periportal zone” (zone 1) are closest to the portal triad and receive the most oxygenated blood, the pericentral zone (zone 3) are closest to the central vein and therefore receive the least amount of oxygenated blood, and the transition zone (zone 2) is in between zone 1 and 3. Due to this separation, each zone of hepatocytes exhibit differing activities. For example, zone 1 hepatocytes are involved in oxidative liver functions such as gluconeogenesis and oxidative metabolism of fatty acids, whereas zone 3 hepatocytes are involved in glycolysis, lipogenesis, and cytochrome P450-mediated detoxification. In some embodiments, the liver organoids disclosed herein exhibit a periportal-like identity resembling the tissue found in the periportal zone of liver lobules, including the functional and cellular marker characteristics of the periportal zone.

The term “% w/w” or “% wt/wt” as used herein has its plain and ordinary meaning as understood in light of the specification and refers to a percentage expressed in terms of the weight of the ingredient or agent over the total weight of the composition multiplied by 100. The term “% v/v” or “% vol/vol” as used herein has its plain and ordinary meaning as understood in the light of the specification and refers to a percentage expressed in terms of the liquid volume of the compound, substance, ingredient, or agent over the total liquid volume of the composition multiplied by 100.

### Stem Cells

The term “totipotent stem cells” (also known as omnipotent stem cells) as used herein has its plain and ordinary meaning as understood in light of the specification and are stem cells that can differentiate into embryonic and extra-embryonic cell types. Such cells can construct a complete, viable organism. These cells are produced from the fusion of an egg and sperm cell. Cells produced by the first few divisions of the fertilized egg are also totipotent.

The term “embryonic stem cells (ESCs),” also commonly abbreviated as ES cells, as used herein has its plain and ordinary meaning as understood in light of the specification and refers to cells that are pluripotent and derived from the inner cell mass of the blastocyst, an early-stage embryo. For purpose of the present disclosure, the term “ESCs” is used broadly sometimes to encompass the embryonic germ cells as well.

The term “pluripotent stem cells (PSCs)” as used herein has its plain and ordinary meaning as understood in light of the specification and encompasses any cells that can differentiate into nearly all cell types of the body, i.e., cells derived from any of the three germ layers (germinal epithelium), including endoderm (interior stomach lining, gastrointestinal tract, the lungs), mesoderm (muscle, bone, blood, urogenital), and ectoderm (epidermal tissues and nervous system). PSCs can be the descendants of inner cell mass cells of the preimplantation blastocyst or obtained through induction of a non-pluripotent cell, such as an adult somatic cell, by forcing the expression of certain genes. Pluripotent stem cells can be derived from any suitable source. Examples of sources of pluripotent stem cells include mammalian sources, including human, rodent, porcine, and bovine.

The term “induced pluripotent stem cells (iPSCs),” also commonly abbreviated as iPS cells, as used herein has its plain and ordinary meaning as understood in light of the specification and refers to a type of pluripotent stem cells artificially derived from a normally non-pluripotent cell, such as an adult somatic cell, by inducing a “forced” expression of certain genes. hiPSC refers to human iPSCs. In some methods known in the art, iPSCs may be derived by transfection of certain stem cell-associated genes into non-pluripotent cells, such as adult fibroblasts. Transfection may be achieved through viral transduction using viruses such as retroviruses or lentiviruses. Transfected genes may include the master transcriptional regulators Oct-3/4 (POU5F1) and Sox2, although other genes may enhance the efficiency of induction. After 3-4 weeks, small numbers of transfected cells begin to become morphologically and biochemically similar to pluripotent stem cells, and are typically isolated through morphological selection, doubling time, or through a reporter gene and antibiotic selection. As used herein, iPSCs include first generation iPSCs, second generation iPSCs in mice, and human induced pluripotent stem cells. In some methods, a retroviral system is used to transform human fibroblasts into pluripotent stem cells using four pivotal genes: Oct3/4, Sox2, Klf4, and c-Myc. In other methods, a lentiviral system is used to transform somatic cells with OCT4, SOX2, NANOG, and LIN28. Genes whose expression are induced in iPSCs include but are not limited to Oct-3/4 (POU5F1); certain members of the Sox gene family (e.g., Sox1, Sox2, Sox3, and Sox15); certain members of the Klf family (e.g., Klf1, Klf2, Klf4, and Klf5), certain members of the Myc family (e.g., C-myc, L-myc, and N-myc), Nanog, LIN28, Tert, Fbx15, ERas, ECAT15-1, ECAT15-2, Tcl1, β-Catenin, ECAT1, Esg1, Dnmt3L, ECAT8, Gdf3, Fth117, Sal14, Rex1, UTF1, Stella, Stat3, Grb2, Prdm14, Nr5a1, Nr5a2, or E-cadherin, or any combination thereof.

The term “precursor cell” as used herein has its plain and ordinary meaning as understood in light of the specification and encompasses any cells that can be used in methods described herein, through which one or more precursor cells acquire the ability to renew itself or differentiate into one or more specialized cell types. In some embodiments, a precursor cell is pluripotent or has the capacity to becoming pluripotent. In some embodiments, the precursor cells are subjected to the treatment of external factors (e.g., growth factors) to acquire pluripotency. In some embodiments, a precursor cell can be a totipotent (or omnipotent) stem cell; a pluripotent stem cell (induced or non-induced); a multipotent stem cell; an oligopotent stem cells and a unipotent stem cell. In some embodiments, a precursor cell can be from an embryo, an infant, a child, or an adult. In some embodiments, a precursor cell can be a somatic cell subject to treatment such that pluripotency is conferred via genetic manipulation or protein/peptide treatment. Precursor cells include embryonic stem cells (ESC), embryonic carcinoma cells (ECs), and epiblast stem cells (EpiSC).

In some embodiments, one step is to obtain stem cells that are pluripotent or can be induced to become pluripotent. In some embodiments, pluripotent stem cells are derived from embryonic stem cells, which are in turn derived from totipotent cells of the early mammalian embryo and are capable of unlimited, undifferentiated proliferation in vitro. Embryonic stem cells are pluripotent stem cells derived from the inner cell mass of the blastocyst, an early-stage embryo. Methods for deriving embryonic stem cells from blastocytes are well known in the art. It would be understood by one of skill in the art that the methods and systems described herein are applicable to any stem cells.

Additional stem cells that can be used in embodiments in accordance with the present disclosure include but are not limited to those provided by or described in the database hosted by the National Stem Cell Bank (NSCB), Human Embryonic Stem Cell Research Center at the University of California, San Francisco (UCSF); WISC cell Bank at the Wi Cell Research Institute; the University of Wisconsin Stem Cell and Regenerative Medicine Center (UW-SCRMC); Novocell, Inc. (San Diego, Calif.); Cellartis AB (Goteborg, Sweden); ES Cell International Pte Ltd (Singapore); Technion at the Israel Institute of Technology (Haifa, Israel); and the Stem Cell Database hosted by Princeton University and the University of Pennsylvania. Exemplary embryonic stem cells that can be used in embodiments in accordance with the present disclosure include but are not limited to SA01 (SA001); SA02 (SA002); ES01 (HES-1); ES02 (HES-2); ES03 (HES-3); ES04 (HES-4); ES05 (HES-5); ES06 (HES-6); BG01 (BGN-01); BG02 (BGN-02); BG03 (BGN-03); TE03 (13); TE04 (14); TE06 (16); UCOl (HSF1); UC06 (HSF6); WA01 (HI); WA07 (H7); WA09 (H9); WA13 (H13); WA14 (H14). Exemplary human pluripotent cell lines include but are not limited to TkDA3-4, 1231A3, 317-D6, 317-A4, CDH1, 5-T-3, 3-34-1, NAFLD27, NAFLD77, NAFLD150, WD90, WD91, WD92, L20012, C213, 1383D6, FF, or 317-12 cells.

In developmental biology, cellular differentiation is the process by which a less specialized cell becomes a more specialized cell type. As used herein, the term “directed differentiation” describes a process through which a less specialized cell becomes a particular specialized target cell type. The particularity of the specialized target cell type can be determined by any applicable methods that can be used to define or alter the destiny of the initial cell. Exemplary methods include but are not limited to genetic manipulation, chemical treatment, protein treatment, and nucleic acid treatment.

In some embodiments, an adenovirus can be used to transport the requisite four genes, resulting in iPSCs substantially identical to embryonic stem cells. Since the adenovirus does not combine any of its own genes with the targeted host, the danger of creating tumors is eliminated. In some embodiments, non-viral based technologies are employed to generate iPSCs. In some embodiments, reprogramming can be accomplished via plasmid without any virus transfection system at all, although at very low efficiencies. In other embodiments, direct delivery of proteins is used to generate iPSCs, thus eliminating the need for viruses or genetic modification. In some embodiment, generation of mouse iPSCs is possible using a similar methodology: a repeated treatment of the cells with certain proteins channeled into the cells via poly-arginine anchors was sufficient to induce pluripotency. In some embodiments, the expression of pluripotency induction genes can also be increased by treating somatic cells with FGF2 under low oxygen conditions.

The term “feeder cell” as used herein has its plain and ordinary meaning as understood in light of the specification and refers to cells that support the growth of pluripotent stem cells, such as by secreting growth factors into the medium or displaying on the cell surface. Feeder cells are generally adherent cells and may be growth arrested. For example, feeder cells are growth-arrested by irradiation (e.g. gamma rays), mitomycin-C treatment, electric pulses, or mild chemical fixation (e.g. with formaldehyde or glutaraldehyde). However, feeder cells do not necessarily have to be growth arrested. Feeder cells may serve purposes such as secreting growth factors, displaying growth factors on the cell surface, detoxifying the culture medium, or synthesizing extracellular matrix proteins. In some embodiments, the feeder cells are allogeneic or xenogeneic to the supported target stem cell, which may have implications in downstream applications. In some embodiments, the feeder cells are mouse cells. In some embodiments, the feeder cells are human cells. In some embodiments, the feeder cells are mouse fibroblasts, mouse embryonic fibroblasts, mouse STO cells, mouse 3T3 cells, mouse SNL 76/7 cells, human fibroblasts, human foreskin fibroblasts, human dermal fibroblasts, human adipose mesenchymal cells, human bone marrow mesenchymal cells, human amniotic mesenchymal cells, human amniotic epithelial cells, human umbilical cord mesenchymal cells, human fetal muscle cells, human fetal fibroblasts, or human adult fallopian tube epithelial cells. In some embodiments, conditioned medium prepared from feeder cells is used in lieu of feeder cell co-culture or in combination with feeder cell co-culture. In some embodiments, feeder cells are not used during the proliferation of the target stem cells.

### Differentiation of PSCs

Known methods for making downstream cell types, such as definitive endoderm, foregut endoderm, ventral foregut endoderm, and hepatic lineages from pluripotent cells (e.g., iPSCs or ESCs) are applicable to the methods described herein. In some embodiments, pluripotent cells are derived from a morula. In some embodiments, pluripotent stem cells are stem cells. Stem cells used in these methods can include, but are not limited to, embryonic stem cells or induced pluripotent stem cells. Embryonic stem cells can be derived from the embryonic inner cell mass or from the embryonic gonadal ridges. Embryonic stem cells can originate from a variety of animal species including, but not limited to, various mammalian species including humans. In some embodiments, human embryonic stem cells are used to produce definitive endoderm or other downstream cell types such as foregut endoderm, ventral foregut endoderm, and hepatic lineages. In some embodiments, iPSCs are used to produce definitive endoderm or other downstream cell types such as foregut endoderm, ventral foregut endoderm, and hepatic lineages. In some embodiments, human iPSCs (hiPSCs) are used to produce definitive endoderm or other downstream cell types such as foregut endoderm, ventral foregut endoderm, and hepatic lineages.

In some embodiments, PSCs, such as ESCs and iPSCs, undergo directed differentiation into embryonic germ layer cells, organ tissue progenitor cells, and then into tissue such as liver tissue or any other biological tissue. In some embodiments, the directed differentiation is done in a stepwise manner to obtain each of the differentiated cell types where molecules (e.g. growth factors, ligands, agonists, antagonists) are added sequentially as differentiation progresses. In some embodiments, the directed differentiation is done in a non-stepwise manner where molecules (e.g. growth factors, ligands, agonists, antagonists) are added at the same time. In some embodiments, directed differentiation is achieved by selectively activating certain signaling pathways in the PSCs or any downstream cells.

In some embodiments, the embryonic stem cells or iPSCs are treated with one or more small molecule compounds, activators, inhibitors, or growth factors for a time that is, is about, is at least, is at least about, is not more than, or is not more than about, 6 hours, 12 hours, 18 hours, 24 hours, 36 hours, 48 hours, 60 hours, 72 hours, 84 hours, 96 hours, 120 hours, 150 hours, 180 hours, 240 hours, 300 hours or any time within a range defined by any two of the aforementioned times, for example 6 hours to 300 hours, 24 hours to 120 hours, 48 hours to 96 hours, 6 hours to 72 hours, or 24 hours to 300 hours. In some embodiments, more than one small molecule compounds, activators, inhibitors, or growth factors are added. In these cases, the more than one small molecule compounds, activators, inhibitors, or growth factors can be added simultaneously or separately.

In some embodiments, the embryonic stem cells or iPSCs are treated with one or more small molecule compounds, activators, inhibitors, or growth factors at a concentration that is, is about, is at least, is at least about, is not more than, or is not more than about, 10 ng/mL, 20 ng/mL, 50 ng/mL, 75 ng/mL, 100 ng/mL, 120 ng/mL, 150 ng/mL, 200 ng/mL, 500 ng/mL, 1000 ng/mL, 1200 ng/mL, 1500 ng/mL, 2000 ng/mL, 5000 ng/mL, 7000 ng/mL, 10000 ng/mL, or 15000 ng/mL, or any concentration that is within a range defined by any two of the aforementioned concentrations, for example, 10 ng/mL to 15000 ng/mL, 100 ng/mL to 5000 ng/mL, 500 ng/mL to 2000 ng/mL, 10 ng/mL to 2000 ng/mL, or 1000 ng/mL to 15000 ng/mL. In some embodiments, concentration of the one or more small molecule compounds, activators, inhibitors, or growth factors is maintained at a constant level throughout the treatment. In some embodiments, concentration of the one or more small molecule compounds, activators, inhibitors, or growth factors is varied during the course of the treatment. In some embodiments, more than one small molecule compounds, activators, inhibitors, or growth factors are added. In these cases, the more than one small molecule compounds, activators, inhibitors, or growth factors can differ in concentrations.

In some embodiments, the ESCs or iPSCs are cultured in growth media that supports the growth of stem cells. In some embodiments, the ESCs or iPSCs are cultured in stem cell growth media. In some embodiments, the stem cell growth media is RPMI 1640, DMEM, DMEM/F12, or Advanced DMEM/F12. In some embodiments, the stem cell growth media comprises fetal bovine serum (FBS). In some embodiments, the stem cell growth media comprises FBS at a concentration that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, or 20%, or any percentage within a range defined by any two of the aforementioned concentrations, for example 0% to 20%, 0.2% to 10%, 2% to 5%, 0% to 5%, or 2% to 20%. In some embodiments, the stem cell growth media does not contain xenogeneic components. In some embodiments, the growth media comprises one or more small molecule compounds, activators, inhibitors, or growth factors.

In some embodiments, pluripotent stem cells are prepared from somatic cells. In some embodiments, pluripotent stem cells are prepared from biological tissue obtained from a biopsy. In some embodiments, the pluripotent stem cells are cryopreserved. In some embodiments, the somatic cells are cryopreserved. In some embodiments, pluripotent stem cells are prepared from PBMCs. In some embodiments, human PSCs are prepared from human PBMCs. In some embodiments, pluripotent stem cells are prepared from cryopreserved PBMCs. In some embodiments, PBMCs are grown on a feeder cell substrate. In some embodiments, PBMCs are grown on a mouse embryonic fibroblast (MEF) feeder cell substrate. In some embodiments, PBMCs are grown on an irradiated MEF feeder cell substrate.

In some embodiments, stem cells are treated with one or more growth factors to differentiate to definitive endoderm cells. Such growth factors can include growth factors from the TGF-beta superfamily. In some embodiments, the one or more growth factors comprise the Nodal/Activin and/or the BMP subgroups of the TGF-beta superfamily of growth factors. In some embodiments, the one or more growth factors are selected from the group consisting of Nodal, Activin A, Activin B, BMP4, Wnt3a or combinations of any of these growth factors. In some embodiments, the stem cells are contacted with Activin A. In some embodiments, the stem cells are contacted with Activin A and BMP4.

In some embodiments, definitive endoderm (DE) can further undergo anterior endoderm pattering, foregut specification and morphogenesis, dependent on FGF, Wnt, BMP, or retinoic acid, or any combination thereof. In some embodiments, human PSCs are efficiently directed to differentiate in vitro into liver epithelium and mesenchyme. It will be understood that molecules such as growth factors can be added to any stage of the development to promote a particular type of hepatic tissue formation. In some embodiments, siRNA and/or shRNA targeting cellular constituents associated with the FGF, Wnt, BMP, or retinoic acid signaling pathways are used to inhibit or activate these pathways.

### Foregut Cells and Liver Organoids

Methods of making liver organoids have been explored previously in, for example, Ouchi et al. “Modeling Steatohepatitis in Humans with Pluripotent Stem Cell-Derived Organoids” Cell Metabolism (2019) 30(2):374-384; Shinozawa et al. “High-Fidelity Drug-Induced Liver Injury Screen Using Human Pluripotent Stem Cell-Derived Organoids” Gastroenterology (2021) 160(3):831-846; PCT Publications WO 2018/085615, WO 2018/191673, WO 2018/226267, WO 2019/126626, WO 2020/023245, WO 2020/069285, and WO 2021/262676, each of which is hereby expressly incorporated by references in its entirety. Disclosure of liver organoid compositions and methods of making thereof are applicable to the human liver organoids (HLOs) described herein.

In some embodiments, pluripotent stem cells, definitive endoderm, foregut endoderm, ventral foregut endoderm, or downstream liver cell types are contacted with a TGF-b pathway inhibitor. In some embodiments, the TGF-b pathway inhibitor comprises one or more of A83-01, RepSox, LY365947, and SB431542. In some embodiments, the cells are not treated with a TGF-b pathway inhibitor. The TGF-b pathway inhibitor provided herein may be used in combination with any of the other growth factors, pathway activators, or pathway inhibitors provided herein.

In some embodiments, pluripotent stem cells, definitive endoderm, foregut endoderm, ventral foregut endoderm, or downstream liver cell types are contacted with an FGF pathway activator. In some embodiments, the FGF pathway activator comprises an FGF protein. In some embodiments, the FGF protein comprises a recombinant FGF protein. In some embodiments, the FGF pathway activator comprises one or more of FGF1, FGF2, FGF3, FGF4, FGF4, FGF5, FGF6, FGF7, FGF8, FGF8, FGF9, FGF10, FGF11, FGF12, FGF13, FGF14, FGF15 (FGF19, FGF15/FGF19), FGF16, FGF17, FGF18, FGF20, FGF21, FGF22, or FGF23. In some embodiments, the cells are not treated with an FGF pathway activator. The FGF pathway activator provided herein may be used in combination with any of the other growth factors, pathway activators, or pathway inhibitors provided herein.

In some embodiments, pluripotent stem cells, definitive endoderm, foregut endoderm, ventral foregut endoderm, or downstream liver cell types are contacted with a Wnt pathway activator. In some embodiments, the Wnt pathway activator comprises a Wnt protein. In some embodiments, the Wnt protein comprises a recombinant Wnt protein. In some embodiments, the Wnt pathway activator comprises Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, BML 284, IQ-1, WAY 262611, or any combination thereof. In some embodiments, the Wnt pathway activator comprises a GSK3 signaling pathway inhibitor. In some embodiments, the Wnt pathway activator comprises CHIR99021, CHIR 98014, AZD2858, BIO, AR-A014418, SB 216763, SB 415286, aloisine, indirubin, alsterpaullone, kenpaullone, lithium chloride, TDZD 8, or TWS119, or any combination thereof. In some embodiments, the Wnt pathway activator is CHIR99021. In some embodiments, the cells are not treated with a Wnt pathway activator. The Wnt pathway activator provided herein may be used in combination with any of the other growth factors, pathway activators, or pathway inhibitors provided herein.

In some embodiments, pluripotent stem cells, definitive endoderm, foregut endoderm, ventral foregut endoderm, or downstream liver cell types are contacted with a VEGF pathway activator. In some embodiments, the VEGF pathway activator comprises one or more of VEGF or GS4012. In some embodiments, the cells are not treated with a VEGF pathway activator. The VEGF pathway activator provided herein may be used in combination with any of the other growth factors, pathway activators, or pathway inhibitors provided herein.

In some embodiments, pluripotent stem cells, definitive endoderm, foregut endoderm, ventral foregut endoderm, or downstream liver cell types are contacted with an EGF pathway activator. In some embodiments, the EGF pathway activator comprises EGF. In some embodiments, the cells are not treated with an EGF pathway activator. The EGF pathway activator provided herein may be used in combination with any of the other growth factors, pathway activators, or pathway inhibitors provided herein.

In some embodiments, pluripotent stem cells, definitive endoderm, foregut endoderm, ventral foregut endoderm, or downstream liver cell types are contacted with ascorbic acid. In some embodiments, the cells are not treated with ascorbic acid. Ascorbic acid as provided herein may be used in combination with any of the other growth factors, pathway activators, or pathway inhibitors provided herein.

In some embodiments, pluripotent stem cells, definitive endoderm, foregut endoderm, ventral foregut endoderm, or downstream liver cell types are contacted with a BMP pathway activator or BMP pathway inhibitor. In some embodiments, the BMP pathway activator comprises a BMP protein. In some embodiments, the BMP protein is a recombinant BMP protein. In some embodiments, the BMP pathway activator comprises BMP1, BMP2, BMP3, BMP4, BMP5, BMP6, BMP7, BMP8a, BMP8b, BMP10, BMP11, BMP15, IDE1, or IDE2, or any combination thereof. In some embodiments, the BMP pathway inhibitor comprises Noggin, RepSox, LY364947, LDN-193189, SB431542, or any combination thereof. In some embodiments, the cells are not treated with a BMP pathway activator or BMP pathway inhibitor. The BMP pathway activator or BMP pathway inhibitor provided herein may be used in combination with any of the other growth factors, pathway activators, or pathway inhibitors provided herein.

In some embodiments, pluripotent stem cells, definitive endoderm, foregut endoderm, ventral foregut endoderm, or downstream liver cell types are contacted with a retinoic acid pathway activator. In some embodiments, the retinoic acid pathway activator comprises retinoic acid, all-trans retinoic acid, 9-cis retinoic acid, CD437, EC23, BS 493, TTNPB, or AM580, or any combination thereof. In some embodiments, the cells are not treated with a retinoic acid pathway activator. The retinoic acid pathway activator provided herein may be used in combination with any of the other growth factors, pathway activators, or pathway inhibitors provided herein.

In some embodiments, pluripotent stem cells are converted into liver cell types via a “one step” process. For example, one or more molecules that can differentiate pluripotent stem cells into DE culture (e.g., Activin A) are combined with additional molecules that can promote directed differentiation of DE culture (e.g., FGF4, CHIR99021, RA) to directly treat pluripotent stem cells.

In some embodiments, the methods further comprise collecting the ventral foregut endoderm cells and differentiating the ventral foregut endoderm cells to liver organoids. In some embodiments, the ventral foregut endoderm cells are cultured until three-dimension (3D) spheroids are formed spontaneously, and the ventral foregut endoderm cells are collected from the spheroids. In some embodiments, the methods further comprise dissociating the spheroids into individual ventral foregut endoderm cells and/or clumps of ventral foregut endoderm cells prior to the differentiating step. In some embodiments, the ventral foregut endoderm cells are collected from the ventral foregut endoderm cell monolayer by dissociating the ventral foregut endoderm cell monolayer into individual ventral foregut endoderm cells and/or clumps of ventral foregut endoderm cells prior to the differentiating step.

In some embodiments, a ventral foregut cell (aka, foregut progenitor, (FG)) is embedded in a basement membrane matrix environment (e.g., Matrigel) as disclosed herein. In some embodiments, where a population pool of organoids is desired, a plurality of individual progenitor cells (e.g. FG cells) are embedded in a single basement membrane matrix environment, where each of the plurality of progenitor cells is from a different donor. In some embodiments, the plurality of different donors is, or is at least, 10, 20, 30, 40, 50, 100, 500, 1000 or more individual donors. In some embodiments, one or more of the plurality of donors can have a desired genetic feature (genotype), for example, a genotype that increases or decrease their risk of a disease (e.g. NAFLD and/or NASH). The plurality of progenitor cells can then be differentiated into organoids (e.g. liver organoids). In some embodiments, the embedded individual FG cell or plurality of individual cells are exposed to an FGF activator, a TGF-beta inhibitor, and a Wnt pathway activator for a period sufficient to promote expansion of the FG cell(s). In some embodiments, the expanded cells form clusters or spheroids. In some embodiments the expanded FG cells are exposed to a retinoic acid pathway activator for a period of time sufficient to differentiate the expanded FG cells into a liver organoid(s). Where a plurality of progenitor cells, each from a different donor, are used, the resulting plurality of liver organoids will each contain cells from only a single donor. In some embodiments, the liver organoids are exposed to hepatocyte growth factor (HGF), oncostatin M (OSM), dexamethasone (DEX), and optionally insulin for a period of time, in amounts and for a time as described herein. In some embodiments, the FGF activator is one described herein, e.g., FGF2, and is in an amount described herein, e.g., 0.5-50 ng/ml, 1-25 ng/mL, 2.5-10 ng/mL, or 5 ng/mL. In some embodiments the TGF-beta inhibitor is one described herein, e.g., A83 01, and is in an amount described herein, e.g., 0.05-5.0 μM, 0.1-2.5 μM, 0.25-1.0 μM, or 0.5 μM. In some embodiments, the Wnt pathway activator is one described herein, e.g., a GSK-3 inhibitor, optionally CHIR99021, and is in an amount described herein, e.g., 0.3-30 μM, 0.6-15 μM, 1.5-6 μM, or 3 μM. In some embodiments, the retinoic acid pathway activator is one described herein, e.g., retinoic acid, and is in an amount described herein, e.g., 0.2-20 μM, 0.4-10 μM, 1.0-4 μM, or 2 μM. In some embodiments, the period sufficient to promote expansion of the FG cell to form expanded FG cells is about 1-8, 2-6, 3-5, or 4 days. In some embodiments, the period of time sufficient to differentiate the expanded FG cells into a liver organoid is about 1-8, 2-6, 3-5, or 4 days. In some embodiments, the liver organoid(s) is cultured in hepatocyte culture medium.

In some embodiments, the FG cells are differentiated from IPSC or other pluripotent stem cells as described herein, or as known in the art. In some embodiments, the FG cell (or plurality of FG cells), is differentiated from an induced-pluripotent stem cell (IPSC) by exposure to Activin A, optionally a BMP pathway activator (e.g., BMP4), a Wnt pathway activator, and an FGF activator. In some embodiments, the IPSC is exposed to Activin A and optionally a BMP pathway activator (e.g., BMP4) for a first period of time, optionally about 1-4, 2-4, or 3 days, and then exposed to the Wnt pathway activator and an FGF activator for a second period of time, optionally about 1-4, 2-4, or 3 days. In some embodiments the preparation of individual FG cells for embedding in a basement membrane matrix includes dissociating clusters of FG cells into single FG cells, and optionally, cryopreserving single FG cells prior to embedding in the basement membrane matrix. In some embodiments, the Activin Activin A is in an amount disclosed herein, e.g, 1-10000 ng/mL, 10-1000 ng/mL, 20-500 ng/mL, 50-200 ng/mL, or 100 ng/mL. In some embodiments the Wnt pathway activator is one described herein, e.g., a GSK-3 inhibitor, optionally CHIR99021, and is in an amount described herein, e.g., 0.3-30 μM, 0.6-15 μM, 1.5-6 μM, or 3 μM. In some embodiments, the FGF activator is one described herein, e.g., FGF4, and is in an amount described herein, e.g., 50-5000 ng/ml, 100-2500 ng/mL, 250-1000 ng/mL, or 500 ng/mL. In some embodiments the liver organoid has a genotype comprising a single nucleotide polymorphism (SNP) variant selected from the group consisting of PNPLA3 rs738409, GCKR-rs1260326, GCKR-rs780094, and TM6SF2-rs58542926. In some embodiments the FG cell is human. In some embodiments, the method further comprises exposing the liver organoid to a fatty acid, optionally oleic acid, and optionally insulin, to generate a steatohepatitis-like liver organoid.

In some embodiments, ventral foregut endoderm cells (aka FG cells) are differentiated into liver organoids. In some embodiments, the methods comprise i) contacting ventral foregut endoderm cells, optionally in the form of spheroids, optionally in the form of individual cells or cell clusters dissociated from spheroids, and/or optionally cells aggregated in a microwell or other apparatus as described herein, with a retinoic acid pathway activator; and ii) contacting the cells of step i) with hepatocyte growth factor (HGF), oncostatin M (OSM), and dexamethasone (DEX), for a period of time thereby differentiating the ventral foregut endoderm cells to liver organoids. In some embodiments, the ventral foregut endoderm cells may be any of the ventral foregut endoderm cells disclosed herein. In some embodiments, the ventral foregut endoderm cells are in the form of spheroids or individual ventral foregut endoderm cells and/or clumps of ventral foregut endoderm cells derived from dissociating the spheroids. In some embodiments, the ventral foregut endoderm cells may be produced by a method that does not involve the use of a xenogeneic basement membrane matrix. In some embodiments, the ventral foregut endoderm cells may be those generally known in the art.

In some embodiments of the methods provided herein, the retinoic acid pathway activator is selected from the group consisting of retinoic acid, all-trans retinoic acid, 9-cis retinoic acid, CD437, EC23, BS 493, TTNPB, and AM580. In some embodiments, the retinoic acid pathway activator is retinoic acid. In some embodiments, the retinoic acid pathway activator is provided at a concentration that is, is about, is at least, is at least about, is not more than, or is not more than about, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 μM, or any concentration within a range defined by any two of the aforementioned concentrations, for example, 1.0-3.0 μM, 1.0-2.0 μM, 2.0-3.0 μM, or 1.5-2.5 μM. In some embodiments, the retinoic acid pathway activator is provided at a concentration of, or of about, 2.0 μM.

In some embodiments of the methods provided herein, the HGF is provided at a concentration that is, is about, is at least, is at least about, is not more than, or is not more than about, 1, 2, 3, 4, 5, 6, 7, 8, 9, 10, 11, 12, 13, 14, 15, 16, 17, 18, 19, or 20 ng/mL, or any concentration within a range defined by any two of the aforementioned concentrations, for example, 1-20 ng/mL, 1-10 ng/mL, 10-20 ng/mL, or 5-15 ng/mL. In some embodiments, the HGF is provided at a concentration of, or of about 10 ng/mL.

In some embodiments of the methods provided herein, the OSM is provided at a concentration that is, is about, is at least, is at least about, is not more than, or is not more than about, 10, 11, 12, 13, 14, 15, 16, 17, 18, 19, 20, 21, 22, 23, 24, 25, 26, 27, 28, 29, or 30 ng/mL, or any concentration within a range defined by any two of the aforementioned concentrations, for example, 10-30 ng/mL, 10-20 ng/mL, 20-30 ng/mL, or 15-25 ng/mL. In some embodiments, the OSM is provided at a concentration of, or of about 20 ng/mL.

In some embodiments of the methods provided herein, the dexamethasone (DEX) is provided at a concentration that is, is about, is at least, is at least about, is not more than, or is not more than about, 50, 60, 70, 80, 90, 100, 110, 120, 130, 140, 150, 160, 170, 180, 190, or 200 nM, or any concentration within a range defined by any two of the aforementioned concentrations, for example, 50-200 nM, 50-100 nM, 100-200 nM, or 50-150 nM. In some embodiments, the dexamethasone is provided at a concentration of, or of about 100 nM.

In some embodiments of the methods provided herein, the cells of step i) and/or step ii) are contacted in a media that further comprises EGF. In some embodiments, the EGF is provided at a concentration that is, is about, is at least, is at least about, is not more than, or is not more than about, 10, 11, 12, 13, 14, 15, 16, 17, 18, 19, 20, 21, 22, 23, 24, 25, 26, 27, 28, 29, or 30 ng/mL, or any concentration within a range defined by any two of the aforementioned concentrations, for example, 10-30 ng/mL, 10-20 ng/mL, 20-30 ng/mL, or 15-25 ng/mL. In some embodiments, the EGF is provided at a concentration of, or of about, 20 ng/mL. In some embodiments, the cells of step i) and/or step ii) are contacted in a media that does not comprise EGF.

In some embodiments of the methods provided herein, the methods further comprise cryopreserving the liver organoids. In some embodiments, cryopreserving the liver organoids comprises slow-freezing or vitrification cryopreservation. In some embodiments, the liver organoids are cryopreserved with chroman 1, emricasan, polyamine, and trans-ISRIB (CEPT). In some embodiments, chroman 1 is provided at a concentration of or of about 50 nM. In some embodiments, emricasan is provided at a concentration of or of about 5 μM. In some embodiments, polyamine is provided at a concentration of or of about 1:1000. In some embodiments, trans-ISRIB is provided at a concentration of or of about 7 μM.

Disclosed herein are the liver organoids produced by the methods disclosed herein, as well as a population organoid panels produced by the methods disclosed herein.

As applied to any of the cells disclosed herein, such as pluripotent stem cells, definitive endoderm, foregut endoderm, ventral foregut endoderm, or liver organoids, the cells may be derived from a patient. In some embodiments, the patient has a liver disease. In some embodiments, the definitive endoderm, foregut endoderm, ventral foregut endoderm, or liver organoids may be derived from pluripotent stem cells, such as embryonic stem cells or induced pluripotent stem cells.

### Gene Editing

In some embodiments, the iPSCs, definitive endoderm cells, anterior foregut spheroids, or organoids are genetically modified or edited according to methods known in the art. For example, gene editing using CRISPR nucleases such as Cas9 are explored in PCT Publications WO 2013/176772, WO 2014/093595, WO 2014/093622, WO 2014/093655, WO 2014/093712, WO 2014/093661, WO 2014/204728, WO 2014/204729, WO 2015/071474, WO 2016/115326, WO 2016/141224, WO 2017/023803, and WO 2017/070633, each of which is hereby expressly incorporated by reference in its entirety.

### Methods of Use

Also disclosed herein are methods for screening. In some embodiments, the methods comprise contacting any of the liver organoids or population organoid panels disclosed herein with a candidate compound or composition (compound of interest), and assessing the effects of the candidate compound or composition on the liver organoid. In some embodiments, the liver organoid is a model for a liver disease, and assessing the effects of the candidate compound or composition on the liver organoid comprises assessing the effects of the candidate compound or composition on the liver disease. In some embodiments, the liver organoid has been produced from cells derived from a subject. In some embodiments, the cells derived from the subject are induced pluripotent stem cells. In some embodiments, the subject has a liver disease.

### Methods of Diagnosis/Prognosis and Treatment

Also disclosed herein are methods of treating, assessing risk, diagnosing, and/or prognosing an individual identified as having a SNP variant GCKR-rs1260326. The subject can be identified as having a SNP variant GCKR-rs1260326 by known methods, e.g., gene sequencing. In some embodiments, the method comprises obtaining or having obtained the HbA1c level of a subject (e.g., from a blood sample) identified as having a SNP variant GCKR-rs1260326; determining that the subject has a reduced risk of and/or a good prognosis for fatty acid liver disease (e.g., NAFLD and/or NASH) if the subject's HbA1c level is less than 5.7%, or determining that the subject has an increased risk of and/or a poor prognosis for fatty acid liver disease (e.g., NAFLD and/or NASH) if the subject's HbA1c level is greater than 6.4%. In some embodiments, a method for treating a subject identified as having a SNP variant GCKR-rs1260326, the comprises: a) obtaining or having obtained the HbA1c level of the subject identified as having a SNP variant GCKR-rs1260326; b) determining that the subject has a reduced risk of and/or a good prognosis for fatty acid liver disease if the subject's HbA1c level is less than 5.7%, or determining that the subject has an increased risk of and/or a poor prognosis for fatty acid liver disease if the subject's HbA1c level is greater than 6.4%; and c) administering to the subject that has an increased risk of fatty acid liver disease having an HbA1c level greater than 6.4% a treatment that results in oxidative uncoupling. In some embodiments, a method for treating a subject comprises: selecting a subject identified as having a SNP variant GCKR-rs1260326 and HbA1c level greater than 6.4%; and administering to the subject a treatment that results in oxidative uncoupling. In some embodiments the treatment that results in oxidative uncoupling comprises a NAD+ precursor, optionally nicotinamide riboside, and nitazoxanide. In some embodiments, the treatment further comprises metformin. In some embodiments, the fatty acid liver disease is NAFLD and/or NASH.

### EXAMPLES

### Example 1: Generation of a Steatohepatitis Human Liver Organoid Panel

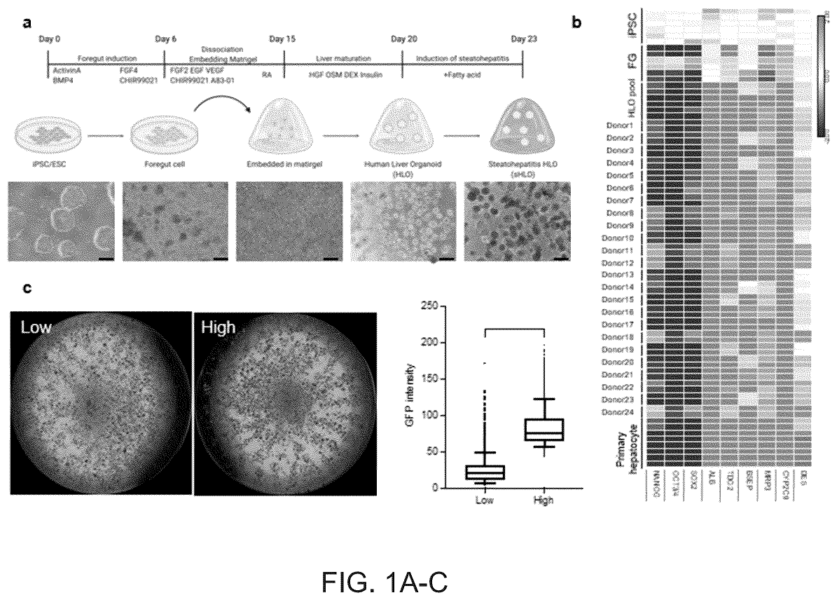

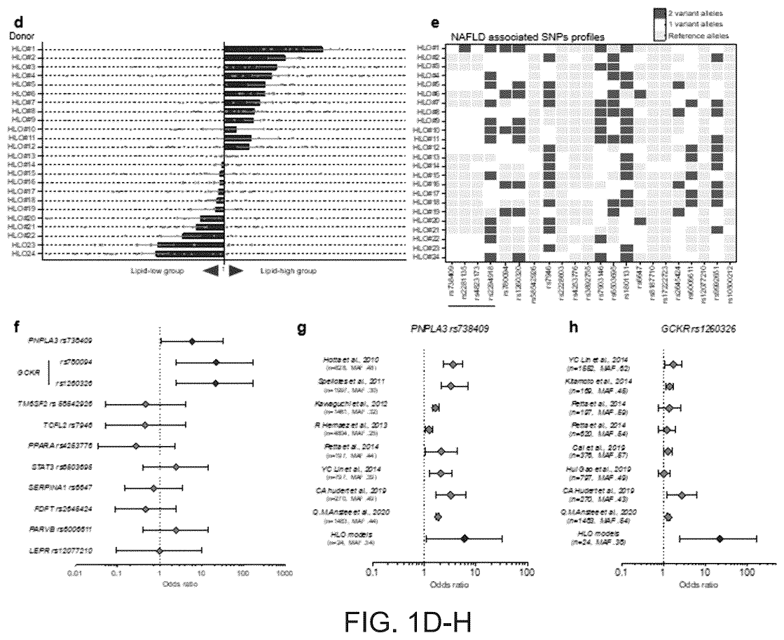

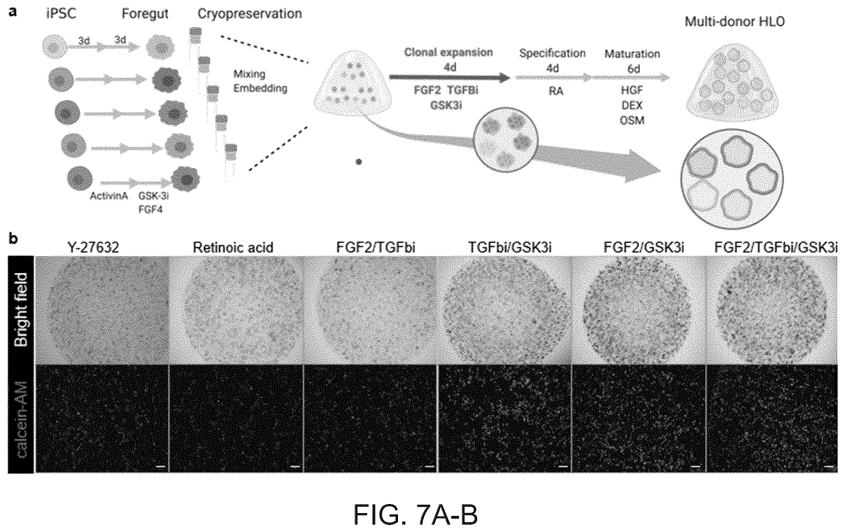

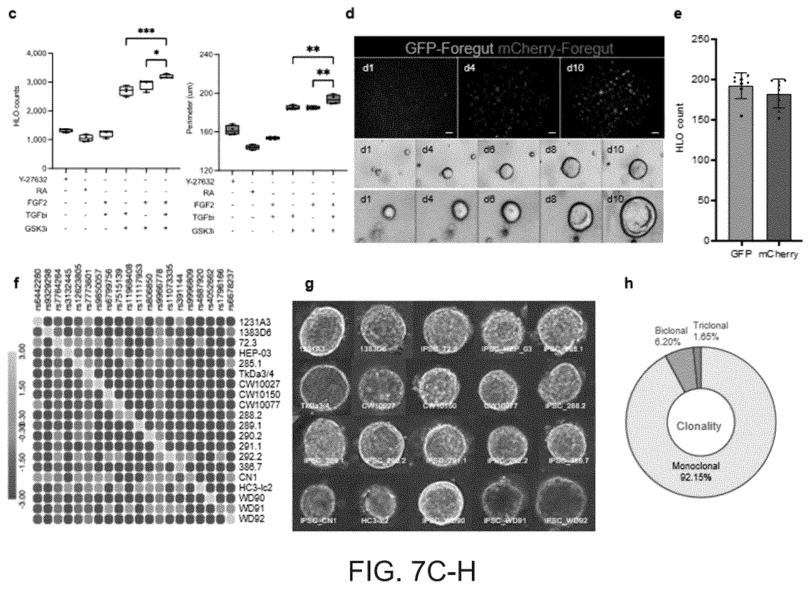

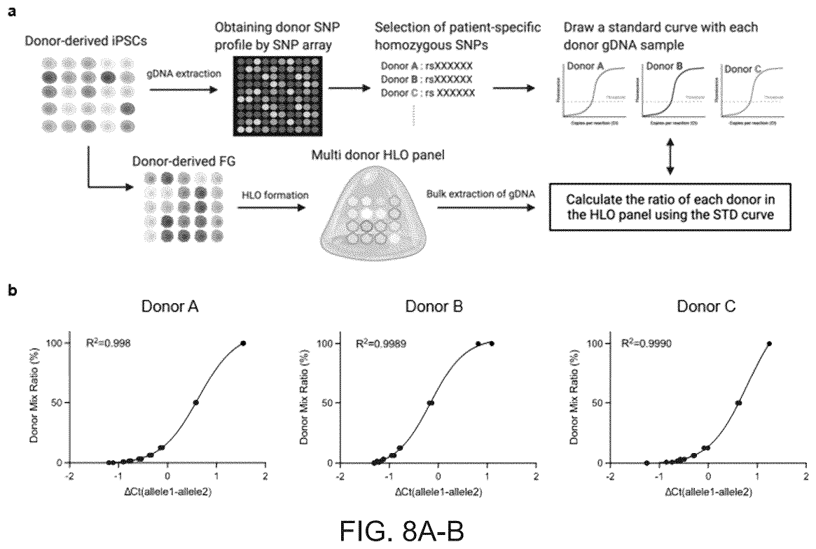

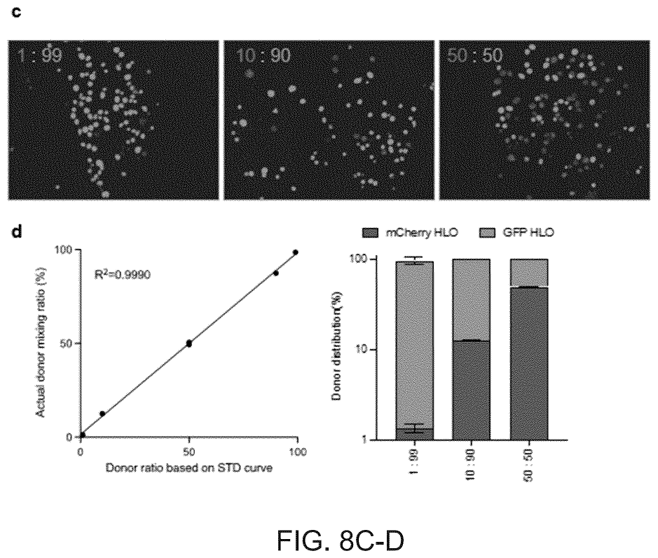

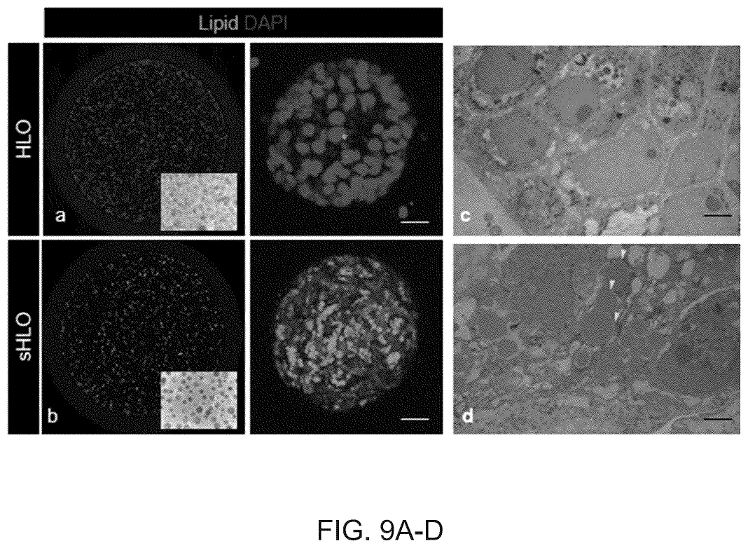

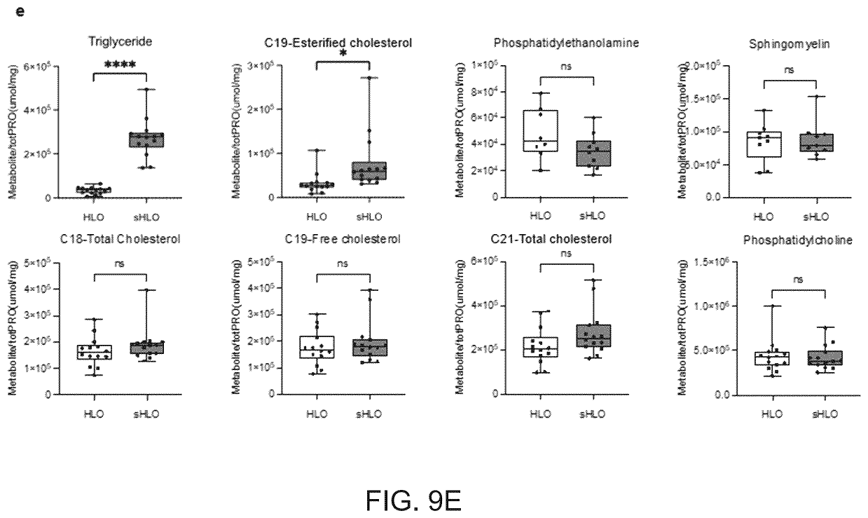

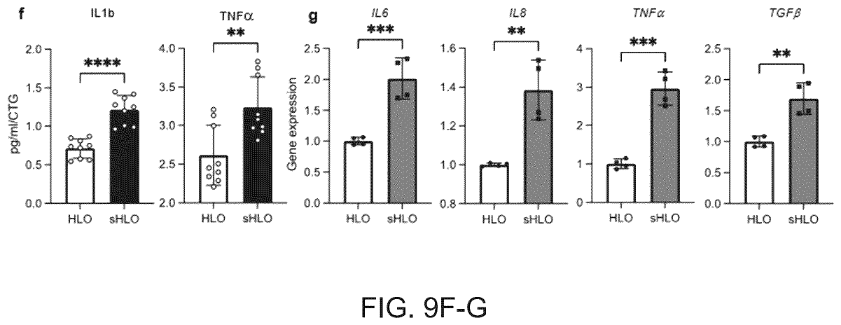

A HLO panel of 24 donor iPSC lines (Supplementary Table 1) was generated to assess the correlation between known significant GWAS SNPs for steatosis, and lipid accumulation phenotypes in our steatohepatitis-like HLO (referred as sHLO for steatohepatitis-like HLO), This number of lines was based on a recent Monte Carlo simulations suggested for an event predicted to occur 1 in 10 patients, a cohort of 24 human iPSC lines would yield a 92% probability that the event will be identified. An iPSC differentiation protocol (schematically depicted in FIG. 1A) was developed for the en masse organoid pooling strategy. The steps for generating a population liver organoid panel include 1) differentiating induced-pluripotent stem cells (IPSCs) from multiple donors into foregut progenitor (FG) (aka ventral foregut) cells by adding ActivinA for the first 3 days of incubation, then treating FG cells with GSK-3 inhibitor and FGF4 on days 4-6, optionally, cryopreserving FG cells after day 6; 2) dissociating clusters of FG cells (e.g. spheroids) into single FG cells; 3) implanting single FG cells from different donors into a single Matrigel environment; 4) treating FG cells with FGF2, TGF-beta inhibitor, and GSK3 inhibitor for 4 days (optionally also with VEGF and EGF) (days 7-10) to promote clonal expansion of FG cell population cells; 5) differentiating into liver organoids by treating with retinoic acid for 4 days (days 11-15); and 6) treating liver organoids with HGF, DEX, and OSM for 6 days (days 16-21). Dissociating iPSC-derived foregut progenitor clusters to single cells prior to embedding in Matrigel, greatly improved the efficiency of generating clonal HLO. The single foregut progenitor cells, furthermore, could be cryopreserved without affecting viability or differentiation capabilities. This, together with improved culturing conditions (the combination of FGF2, TGFb inhibitor and GSK3 inhibitor was the most efficient condition for HLO formation and HLO growth) (FIG. 7A-C), increased the efficiency, and shortened the time frame required, for generating clonal HLOs (FIG. 1A). A pool of 20 cryopreserved donor foregut (FG) progenitors was validated (FIG. 7D-H) to show en masse generation of single donor-derived HLOs. Validation included demonstrations that HLOs formed from single donor cells had: (a) distinct but comparable clonal dynamics of organoid development (FIG. 7D-E); (b) discernable identity based on unique SNP PCR genotyping of the donors (FIG. 7F) and comparable morphology (FIG. 7G); and (c) lack of organoid chimerism where >90% of HLOs carrying single donor-derived SNPs (FIG. 7H). Donor identification was based on unique SNP profiles from microarray analysis, details shown in FIG. 8. To generate a NAFLD phenotype, exposing HLOs to the common fatty acid, oleic acid (OA), induced steatohepatitis-like pathologies of lipid accumulation and inflammation (FIG. 9).

### Example 2: a Steatohepatitis Human Liver Organoid Panel Informs Known Genotype-Phenotype Associations for Non-Alcoholic Fatty Liver Disease (NAFLD)

Then it was assessed to see if common genomic variants associated with NAFLD can inform pathological phenotype of steatohepatitis in the pooled panel of 24 donor HLO model. Targeted transcriptomic analysis of the differentiated pooled 24 FG progenitors showed relatively minimal donor-dependent variations of hepatic gene expression, which were distinct from iPSC and FG, but similar to primary hepatocytes (FIG. 1B). Expression of the markers in the clonally-differentiated HLOs, moreover, were consistent with the individual donor-derived HLOs (FIG. 1B). Although there were some variations amongst individual donor-derived HLO in the expression of genes encoding ATP-binding cassette transporters for drug clearance (BSEP=ABCB11; MRP3=ABCC3), expression of the key hepatic markers, albumin (ALB), a member of cytochrome P450 superfamily CYP2C9, and tryptophan 2.3-dioxygenase TDO2, were comparable (FIG. 1B).

Next, the lipid accumulation phenotype of the 24 pooled clonal HLOs was analyzed. OA-induced sHLOs, quantified by live imaging BODIPY lipid droplet staining and image-based analyses, indicated that lipid-low accumulators can be distinguished from lipid-high accumulators (FIG. 1C). When lipid droplet accumulation was linked to the decoded HLOs, lipid-high and lipid-low phenotypes were associated with donor-specific HLOs (FIG. 1D). Importantly, sHLOs generated from the same donor demonstrated reproducible lipid accumulation abilities.

The en masse steatosis quantification strategy was leveraged to determine whether the organoid based genotype-phenotype association predicts reported common NAFLD-associated SNP genotypes (FIG. 1E). Each donor carried multiple SNPs (FIG. 1E) with odds ratio (OR) analyses (combination of 2 and 1 alleles) showing strong correlations between HLO lipid accumulation phenotype and the most reported risk SNPs, specifically, the PNPLA3-rs738409¹⁸, and controversial GCKR-rs780094 and -rs1260326 risk alleles (FIG. 1F). For other reported SNPs (FIG. 1E), calculated ORs were statistically insignificant in the HLO model system (FIG. 1F; not shown) including the TM6SF2-rs58542926 risk alleles. Since the HLO sample size is small, very low allelic frequency of some of these known SNPs may not be readily captured (e.g. for TM6SF2-rs58542926, frequency T=0.065365, GnomAD exome).

It was notable that none of the 24 donors carried 2 alleles of the well established PNPLA3-rs738409 risk SNP, yet OR was still statistically significant (FIG. 1F) and overlapped with the significant OR calculated for multiple clinical studies (FIG. 1G). Interestingly, in the HLO model, the OR of GCKR-rs1260326 was as significant as PNPLA3-rs738409. Clinical significance of GCKR-rs1260326, in striking contrast, ranged from relatively modest to not significant and had calculated OR decidedly less significant than for the HLO model (FIG. 1H). Collectively, these results indicate that the sHLO model offer an unique human-based system to evaluate pathophysiological significance of potential risk SNPs and, importantly, can inform steatotic genotype-phenotype correlations for NAFLD/NASH.

### Example 3: GCKR Rs1260326 Risk Variant TT (p.Pro446Leu) Increase Susceptibility to De Novo Lipid Accumulation in HLO

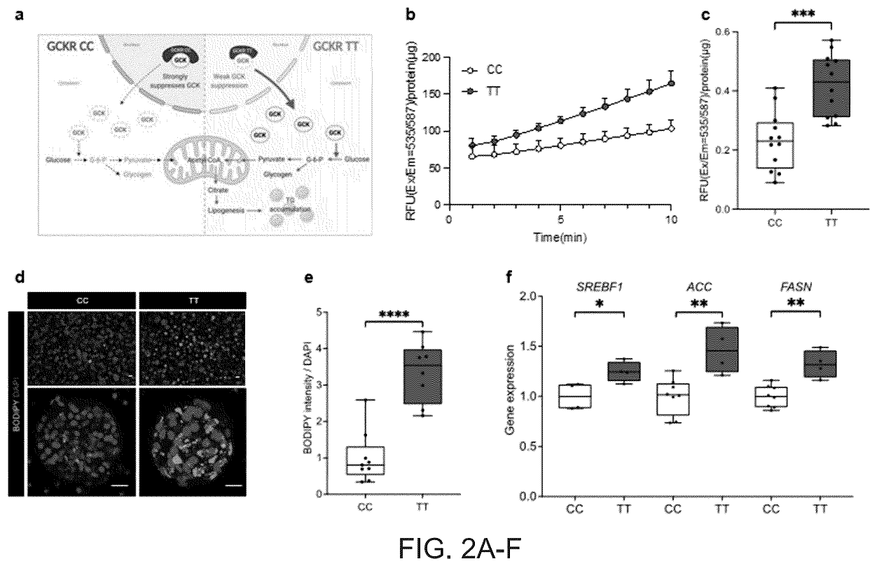

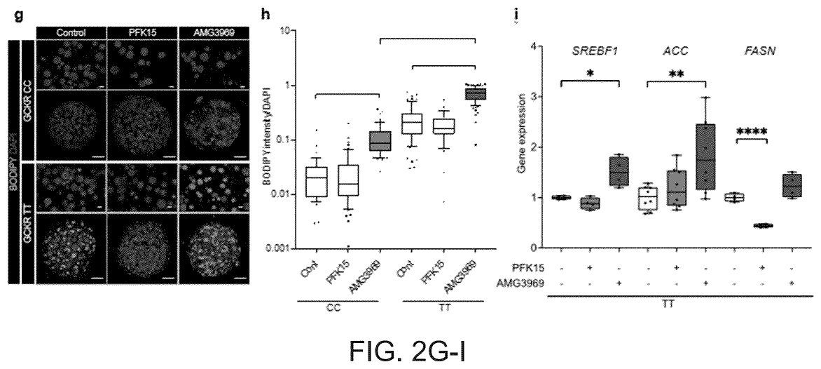

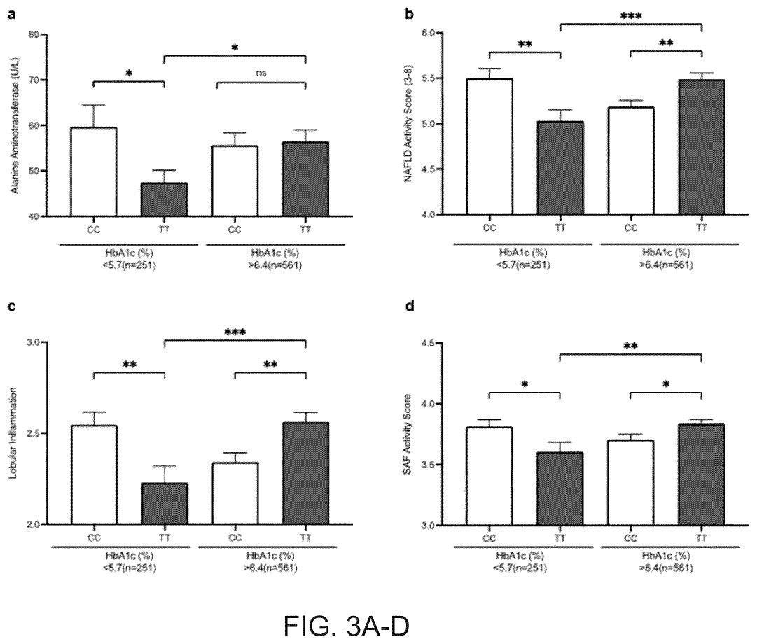

GCKR-rs1260326 is an exonic, functional, SNP, c.1337C>T, in which the C to T substitution alters the proline at position 446 to leucine (p.Pro446Leu), while GCKR-rs780094, in strong linkage disequilibrium with rs1260326, is intronic and non-functional. In the liver, GCKR competes with glucose for binding to GCK and, upon binding, inactivates GCK, in part by retaining GCK in the nucleus (schematically depicted in FIG. 2A). The GCKR-rs1260326 TT variant, with reduced ability to bind GCK22, has been proposed to constitutively activate hepatic glucose uptake and glycolysis with the consequent generation of excess acetyl-CoA, which is normally a rate-limiting substrate for lipogenesis.

To assess the pathogenesis of GCKR-rs1260326 TT risk variant (hereafter referred to as GCKR-TT), independent of PNPLA3-rs738409 GG risk variant, in our HLO model, donor iPSC lines #001 and #006 (FIG. 1B-E, FIG. 14) and iPSC line #29 (FIG. 14) were selected for further analyses, comparing to four GCKR-rs1260326 CC non-risk variant (GCKR-CC) iPSC lines #7, #20, #24 (FIG. 1B-E, FIG. 14) and #25 (FIG. 14). GCK activities in the GCKR-TT HLOs (hereafter refer to as TT HLO) demonstrated remarkable and consistently higher GCK activity than GCKR-CC HLOs (hereafter refer to as CC HLO; FIG. 2B-C). Next, it was evaluated whether the higher GCK activities enhanced de novo lipogenesis (DNL) in the HLO models. Steady-state hepatic lipid metabolism under standard high glucose and high insulin culture conditions without exogenous fatty acid loading required prolonged culturing time and, thus, HLOs were analyzed at Day 30. BODIPY imaging analysis showed that TT HLOs generated and accumulated significantly more lipid droplets compared to CC HLOs (FIG. 2D-E), with enhanced expression of lipogenesis genes SREBP1 (Sterol Regulatory Element-Binding Protein 1), ACC (Acetyl-CoA Carboxylase Alpha) and FASN (Fatty Acid Synthase) (FIG. 2F). Thus, organoids carrying the GCKR-risk variant TT have increased GCK enzyme activities and lipid droplet formation, concordant with increased expression of genes associated with de novo lipogenesis.

To further evaluate the pathogenesis of dysfunctional GCK-GCKR binding, HLOs were treated with PFK15, a 6-phosphofructo-2-kinase (PFKB3) inhibitor, and AMG3969, which disrupts GCK-GCKR binding, and evaluated effects on de novo fat accumulation (FIG. 2G-I). Inhibitory effects of PFK15 should increase Fructose 6-phosphate (F6P) which consequently suppresses GCK activity and inhibits hepatic glycolysis, while disrupting the binding of GCK to GCKR by AMG3969 treatment should result in release and migration of GCK into the cytoplasm from the nucleus, independent of glucose availability, thus enhancing glycolysis and lipogenesis. As predicted, PFK15 inhibition of GCK activity in HLO inhibited lipid accumulation, although this was distinctly more obvious with TT HLO which have higher basal lipid accumulation profiles (FIG. 2G-H). Inhibition of GCK-GCKR complex formation by AMG3969, on the contrary, significantly enhanced lipid droplet accumulation in both non-risk CC HLO and risk TT HLO (FIG. 2G-H). Notably, the maximum lipid droplets detected in CC HLO was consistently below untreated TT HLO (FIG. 2H). For TT HLO, DNL regulatory gene expression was evaluated to determine if the lipid accumulation caused by inhibiting GCK and GCKR actions could be due to glycolysis mediated lipogenesis (FIG. 2I). As shown, SREBP1 and ACC were consistently and significantly increased in AMG3969-treated TT HLOs and FASN was suppressed by PFK15 treatment. Collectively, the results suggest that, in HLO models, the GCKR-rs1260326 risk variant, independent of PNPLA3-rs738409, delineated GCK activities with the GCKR-TT HLOs functionally associated with excessive fat accumulation phenotype through DNL mechanisms, in the absence of exogenous fat-induced lipogenesis.

### Example 4: GCKR-Rs1260326 TT Risk Allele Enhanced Lobular Inflammation in NAFLD Patients with Type 2 Diabetes

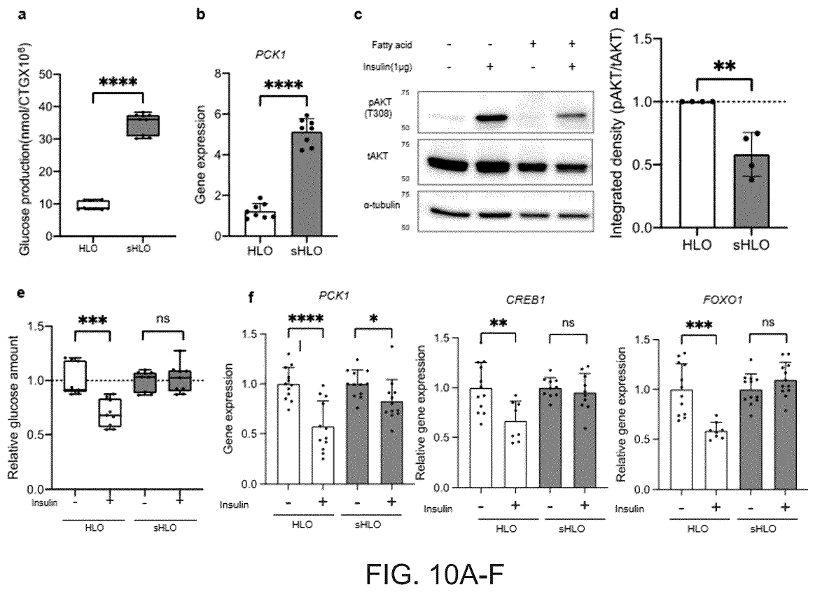

The discrepant metabolic impact of GCKR-rs1260326 TT risk variants between the sHLO model and highly variable clinical reports, raised the question whether other co-morbid metabolic traits, such as onset of diabetes-like symptoms including insulin resistance, should be taken into consideration when evaluating clinical impacts of GCKR-rs1260326 risk alleles. Indeed, the cultured conditions (high glucose and high insulin) for the HLO models are most consistent with NAFLD/NASH and T2D phenotypes and explains the blunted responses to insulin under steatotic conditions, independent of risk variant status (FIG. 10). The significant lipid droplet formation induced in sHLO perturbed gluconeogenesis (FIG. 10A-B) and impaired response to insulin, in terms of signaling, uptake of glucose, regulation of gene expression (FIG. 10C-F), reminiscent of patients with NASH who show hepatic insulin resistance.

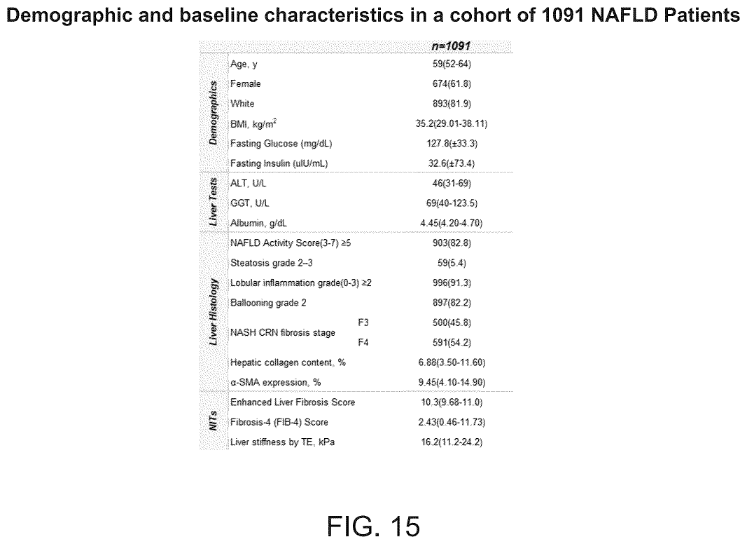

For in vivo assessments, a retrospective analysis was performed on a cohort of 1091 adults diagnosed with NAFLD (STELLAR-3 trial, NCT03053050, and ATLAS-trial, NCT03449446) for which demographic, biomarkers and liver histology of clinical samples were available (FIG. 15). Demographics were predominantly caucasian, middle-aged, females with high BMI in obese ranges.

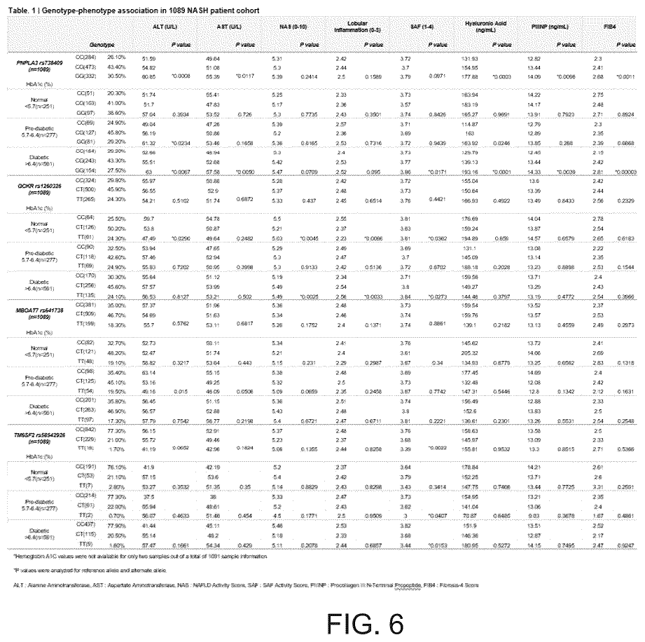

Correlation analyses between NAFLD clinical parameters and genetic risk variants in the cohort focused on GCKR-rs1260326 risk variant compared to the most prevalent risk variants in PNPLA3, MBOAT7 and TM6SF2 (FIG. 6). Measurements include accepted markers of liver injury (ALT, alanine aminotransferase; AST, aspartate aminotransferase), histological grading of NAFLD/NASH pathology (NAFLD Activity Score, NAS; Lobular Inflammation, LI; steatosis, activity, fibrosis, SAF Activity Score, for ballooning and LI) and fibrotic indications (hyaluronic acid; procollagen III N-terminal propeptide, PIIINP; fibrosis-4, FB-4). The well established pathogenic PNPLA3-rs738409, GG risk variant (c.444C>G, p.I148M), as expected, is associated with higher AST and ALT levels (although still within normal ranges), as well as significantly higher PIIINP and hyaluronic acid concentrations (above normal), when compared to the reference CC variant (FIG. 6). The same biomarkers, however, were comparable between reference and risk variants in GCKR, MBOAT7 and TM6SF2. For all variants, histological analyses were within pathological ranges and were indistinguishable (FIG. 6).

It is of note that fasting glucose (normal: <99 mg/dL; prediabetes: 100-125 mg/dL; diabetes: >126 mg/dL) and fasting insulin levels (normal: <25 uIU/ml) were above normal ranges in all patients, irrespective of variants, consistent with indications of insulin resistance (FIG. 14). Therefore, additional association analysis were performed with hemoglobin A1c (HbA1c) data available for 1089 of the 1091 subjects (FIG. 6). HbA1c is a common measurement in T2D diagnosis. HbA1c measures glycated hemoglobin and, unlike fasting glucose, reflects a weighted average of blood glucose levels in the preceding 2-4 months. Specifically, HbA1c cutoff values recommended for adults by the CDC (Center for Disease Control), USA, were used, where below 5.7% is within normal ranges; 5.7%-6.4%, pre-diabetic; and >6.4%, diabetic. Analyses showed that when HbA1c values were in the diabetic>6.4% ranges for PNPLA3-rs728409, the GG risk variant (compared to reference CC) was significantly associated with increased ALT and AST, increased SAF activity score and fibrotic markers, but histological NAS and Lobular inflammation (LI) were not significantly different (FIG. 6).

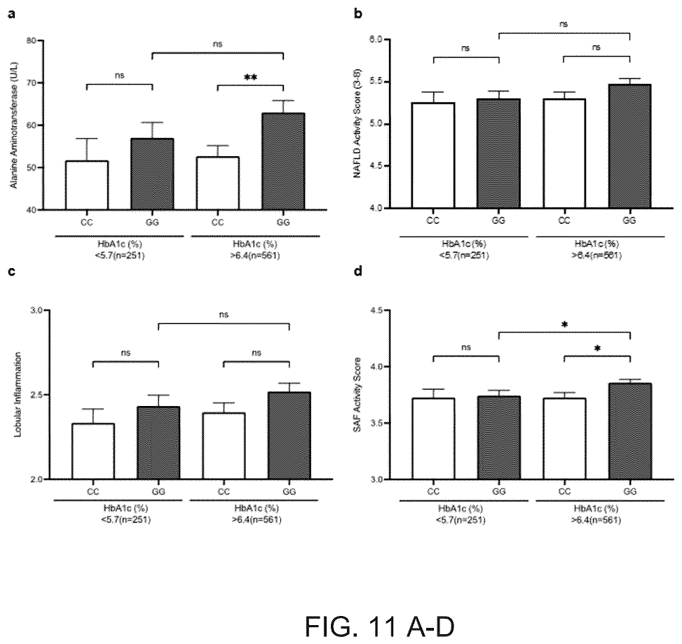

Intriguingly, in patients carrying the GCKR-TT risk variant, ALT, NAS, LI and SAF activity scores were significantly better (i.e. lower scoring) than non-risk reference GCKR-CC when HbA1c values were within normal<5.7% ranges (FIG. 6, FIG. 3A-D). When HbA1c values were in the diabetic>6.4% ranges, scoring was higher (i.e. indicative of worsened pathology) in GCKR-TT versus GCKR-CC cohorts (FIG. 3B-D). This enhanced worsening of NAFLD/NASH inflammatory pathology was most pronounced in the GCKR-TT cohort, when comparing the non-diabetic HbA1c<5.7% to the diabetic HbA1c>6.7% sub-cohorts (FIG. 3). In contrast, for the GCKR-CC non-risk variant, diabetic HbA1c>6.4% conditions did not worsen inflammatory pathology but trended towards improved (lower) scores (FIG. 3). In sum, based on differential HbA1c values, the GCKR-TT risk variant appear to confer striking and unexpected inverse risks for inflammatory pathology (FIG. 3), unique trends not observed with the other genetic risk variants evaluated (FIG. 6), such as, for example, for the PNPLA3-rs728409 GG risk variant (FIG. 11). Thus, for patients carrying the GCKR-TT risk variant, HbA1c measurements may have prognostic value for delineating severity of NAFLD-associated inflammatory pathology.

### Example 5: Mitochondrial Dysregulation is Associated with GCKR TT Related Metabolic Assaults

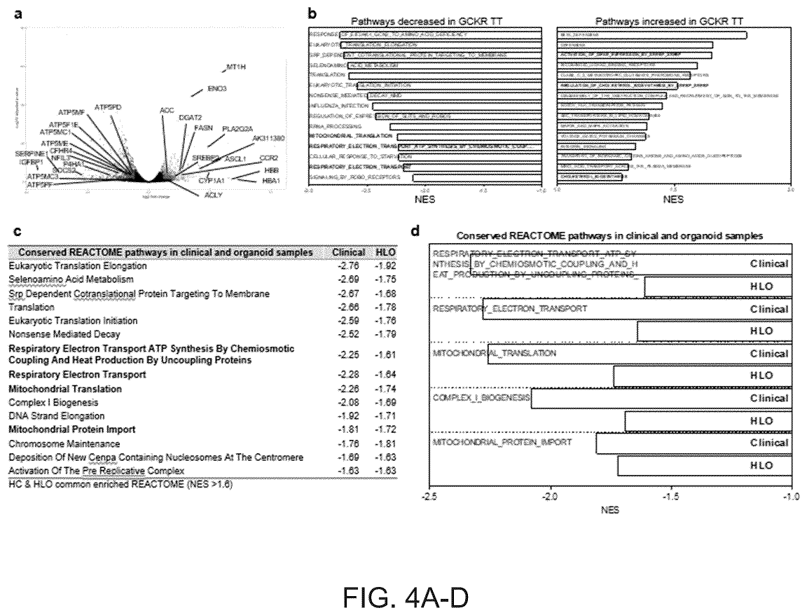

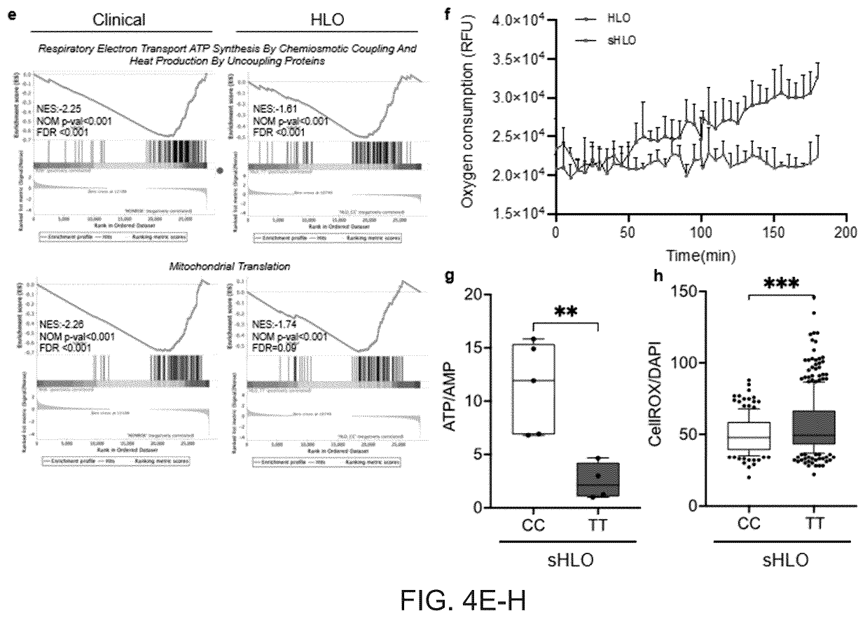

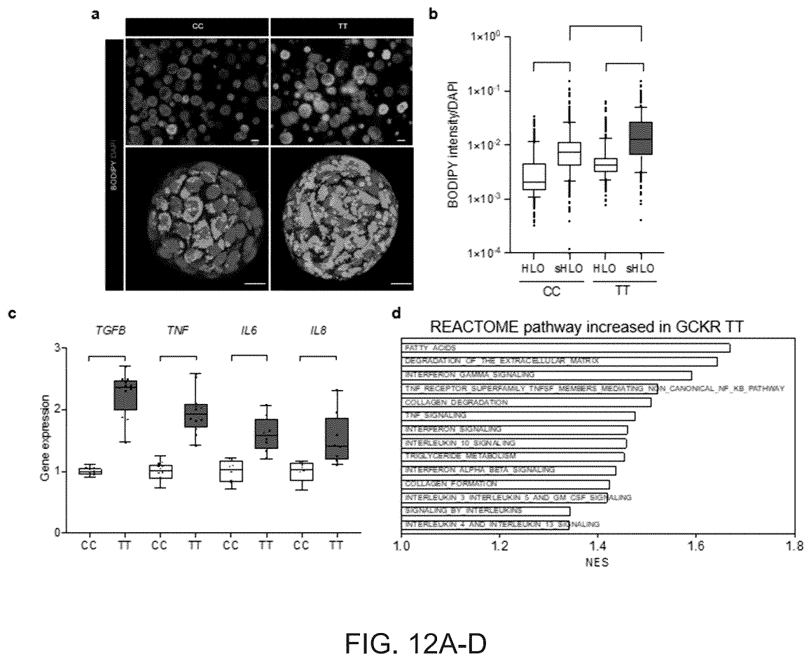

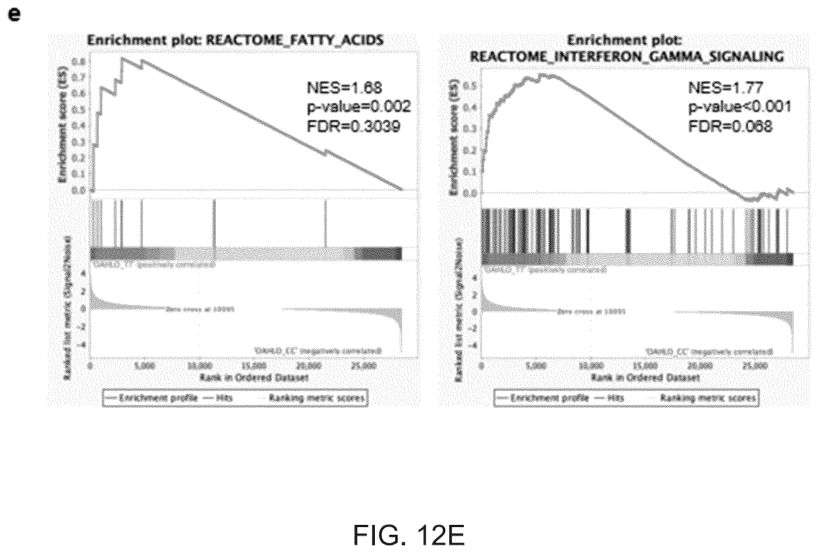

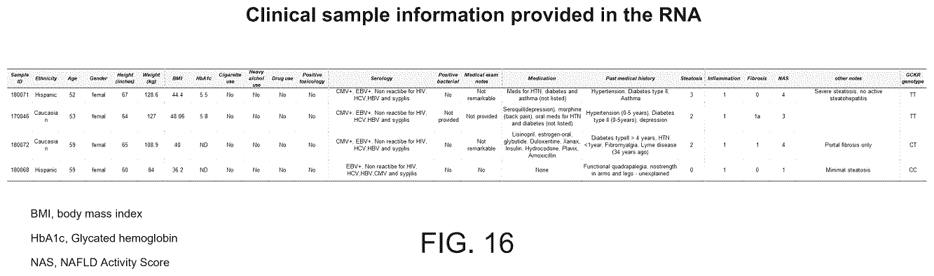

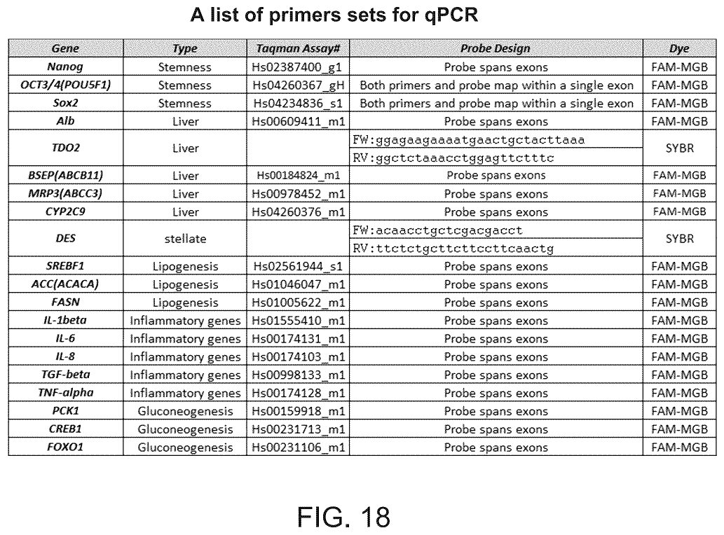

To assess GCKR TT cellular impacts, an unbiased transcriptomic analysis of available RNA-seq datasets from genotyped patient hepatocyte samples (FIGS. 15-16) was performed. In GCKR TT NASH hepatocytes compared to hepatocytes carrying GCKR CC or CT, differential expression gene (DEG) analyses by edgeR (FC>1.50, FDR<0.05) revealed upregulated genes included lipogenic genes (FIG. 4A), similar to the HLO models (FIG. 12; FIG. 18). Intriguingly, down-regulated genes included a cluster encoding the multiple subunits of the mitochondrial ATP synthase (FIG. 4A), a membrane multimeric complex which utilize the electrochemical proton gradient during oxidative phosphorylation to catalyze ATP synthesis from ADP.

The dysregulation of mitochondrial gene sets was also identified by GSEA (gene set enrichment analysis)-REACTOME analysis (FIG. 4B-C). While upregulated pathways included inflammatory gene sets (FIG. 4B), which had been observed in the sHLO models (FIG. 12; FIG. 18), several mitochondrial related pathways were downregulated with NES values amongst the top downregulated REACTOME pathways (FIG. 4C). Strikingly, when comparing these TT NASH hepatocyte samples to TT HLO models, a comparable subset of GSEA-REACTOME pathways were significantly downregulated (FIG. 4C), of which mitochondrial-related REACTOME pathways were the most prominent (FIG. 4D). The down-regulation of the respiratory electron transport ATP synthesis pathways (FIG. 4C-D), moreover, were supported by Enrichment Plot analyses demonstrating that the differences in regulation of gene sets were significant between risk TT and non-risk CC with p<0.001, for both NASH hepatocytes and HLO models (FIG. 4E).

Given the strong transcriptomic indications of mitochondrial dysregulation associated with GCKR TT risk variant compared to non-risk GCKR CC, and strong correlation between clinical TT NASH hepatocytes and the HLO models, the next step was to verify if mitochondrial functions were perturbed in the HLO models. Normally, mitochondrial aerobic respiration relies on electron transfer and a proton gradient to drive ATP (adenosine triphosphate) production, with ROS (reactive oxygen species) as natural by-products which is tightly controlled. It was hypothesized, in the HLO systems, that mitochondrial dysregulation is a consequence of enhanced oxidant stress created by chronic ROS production, impacted by the GCKR TT variant and exacerbated by fatty acid accumulation.

It was shown that oxygen consumption rates (OCR), determined by a fluorescence-based assay, were significantly compromised in TT sHLO compared to TT HLO (FIG. 4F). Further, ATP/AMP ratios determined by intracellular metabolite profiling, was dramatically reduced in TT sHLO compared to CC sHLO (FIG. 4G). These results were consistent with enhanced ROS, quantified by live cell staining, in TT sHLO compared to CC sHLO (FIG. 4H). Hence the HLO and sHLO models readily revealed mitochondrial dysregulation, driven by genetic GCKR-TT risk factor and exogenous fatty acid perturbations.

### Example 6: NR and NTZ, but not Metformin, Mitigates Mitochondrial Dysfunctions of Risk TT sHLO and Reduce Inflammatory Gene Expression

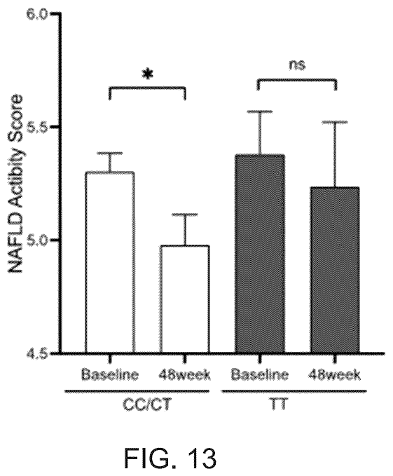

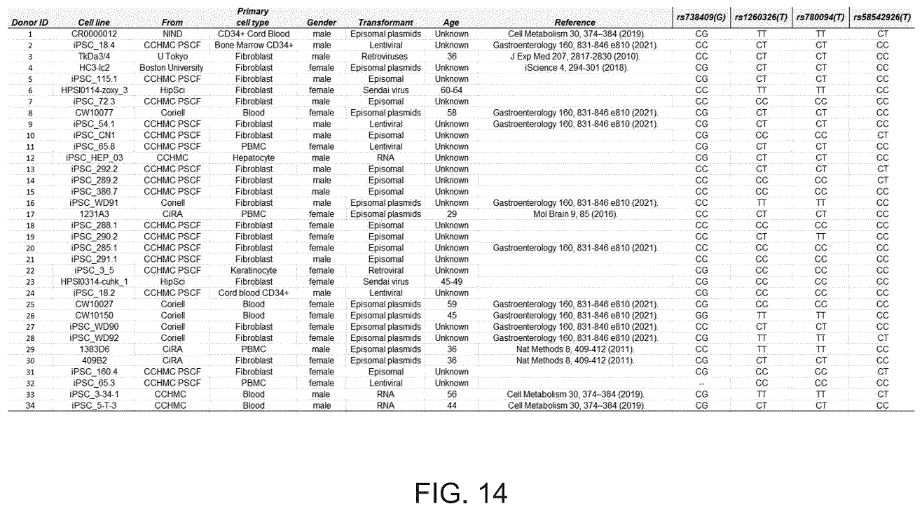

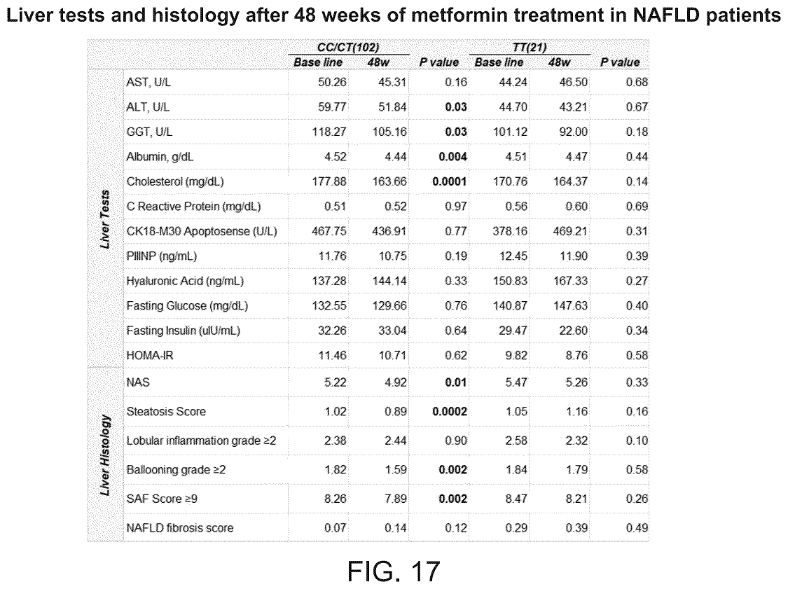

Metformin, the first line of medication used to treat T2D associated with obesity, has been shown to improve mitochondrial respiratory activities via the AMPK pathway in mouse models²⁵. However, patients carrying GCKR TT did not show improvement in multiple phenotypic measurements after 48 weeks of metformin treatment, in contrast to patients carrying GCKR CC or CT (FIG. 13, FIG. 17). Hence, it was inferred that in vivo, metabolic dysfunction associated with GCKR TT, including potential mitochondrial dysregulation, is unlikely to improve with metformin treatment. To determine if the HLO model reflected in vivo observations, the effects of metformin treatment in the sHLO system were evaluated and also assessed whether controlling elevated ROS in TT sHLO can better modulate the undesirable effects of excessive fatty acid accumulation.

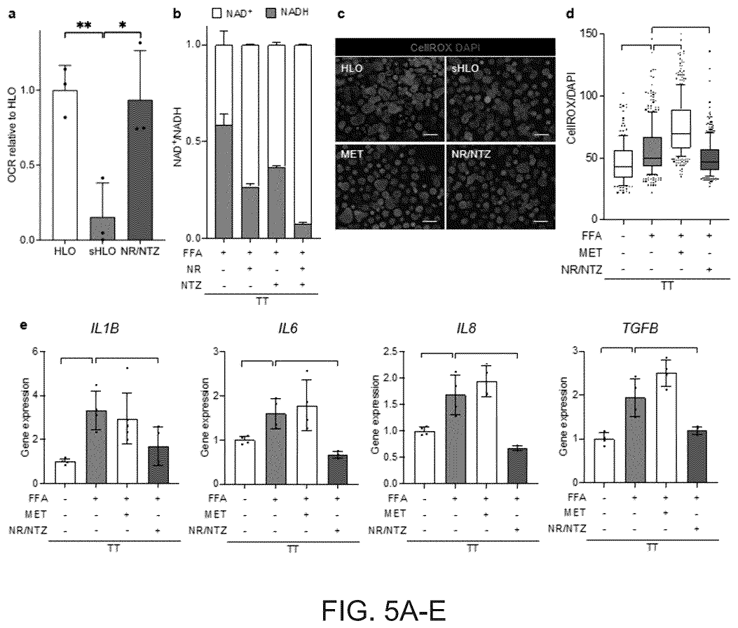

Among the mechanisms to control unwanted generation of ROS, the oxidative phosphorylation process was focused on. Specifically, TT sHLO was supplemented with a NAD+ precursor, nicotinamide riboside (NR), in combination with nitazoxanide (NTZ), a FDA-approved anti-parasitic and anti-viral drug recently shown to possess mitochondrial uncoupling, and respiration-enhancing, activities. The effects of NR and NTZ in our TT HLO systems were demonstrated. Fatty acid exposure (TT sHLO) dramatically reduced OCR, which NR and NTZ co-treatment reversed (FIG. 5A). This restoration of energy consumption in TT sHLO to comparable levels detected in TT HLO is due to synergistic effects of NR and NTZ in increasing NAD+ availability (FIG. 5B). NAD+, the metabolic co-factor involved in redox reactions, is known to protect hepatocytes from the harmful effects of ROS²⁸.

It was confirmed that ROS production was significantly higher in TT sHLO than TT HLO (FIG. 5C-D). Strikingly, metformin treatment did not reduce ROS production and, on the contrary, enhanced ROS in the presence of fatty acid induction. In contrast to metformin, co-treatment with NR and NTZ dramatically decreased ROS formation (FIG. 5C-D) with striking concomitant suppression of fatty acid-induced inflammatory genes (FIG. 5E). Collectively, the susceptibility to significant mitochondrial dysfunction conferred by the GCKR TT risk variant and exacerbated by fatty acid exposure, can be mitigated by oxidative uncoupling mechanisms that permits adaptation to increased fatty acid supply while conferring protection against oxidant stress.

### Example 7: Materials and Methods

iPSC cell lines and cell culture. Human iPSC lines used in this study is summarized in Supplementary Table 1. Patient cells, where applicable, were obtained with consent in compliance with ethics guidelines (Institutional Review Board, Cincinnati Children's Hospital Medical Center) and reprogrammed into iPSC by CCHMC Pluripotent Stem Cell Facility. All human iPSC lines were maintained as described previously. Briefly, undifferentiated hiPSCs were cultured on Laminin 511E8-fragment (Nippi, Japan) coated dishes in Stem Fit medium (Ajinomoto Co, Japan) with 100 ng/ml bFGF (R&D Systems, MN, USA) at 37° C. in 5% CO₂ with 95% air.

Induction and cryopreservation of the foregut. hiPSCs were differentiated into foregut using previously described methods. In brief, hiPSCs were detached by Accutase (Thermo Fisher Scientific Inc., MA, USA) and were seeded on Laminin coated tissue culture plate with 50,000 cells/cm2. Medium was changed to RPMI 1640 medium (Life Technologies) containing 100 ng/mL Activin A (R&D Systems) and 50 ng/mL bone morphogenetic protein 4 (BMP4; R&D Systems) at day 1, 100 ng/mL Activin A and 0.2% fetal calf serum (FCS; Thermo Fisher Scientific Inc.) at day 2, and 100 ng/mL Activin A and 2% FCS at day 3. On Day 4-6, cells were cultured in Advanced DMEM/F12 (Thermo Fisher Scientific Inc.) with B27 (Life Technologies) and N2 (Gibco, CA, USA) containing 500 ng/ml fibroblast growth factor 4 (FGF4; R&D Systems) and 3 μM CHIR99021 (Stemgent, MA, USA). Cells were maintained at 37° C. in 5% CO₂ with 95% air and the medium was replaced daily. The foregut cells were detached by Accutase and then frozen in Cell Banker 1 (Nippon Zenyaku Kogyo Co., Ltd., Japan). The cells were stored at −150 C.

Generation of HLO and pooled organoid panel. The frozen foregut cells were thawed quickly and then centrifuged at 800 rpm for 3 minutes. Cells were suspended with Matrigel™ matrix (Corning Inc., NY, USA) on ice to achieve a final concentration of 750,000 cells/mL. Details of analysis of the pooled organoid panel are described in Extended data FIG. 3a. In brief, the frozen foregut cells derived from each iPSC cell line were mixed and resuspended in Matrigel on ice. The mixture of cells and Matrigel was embedded in 50 μl drop on the dishes in advanced DMEM/F12 with 2% B27, 1% N2, 10 mM HEPES, 1% Glutamax, 1% Pen/Strep, 5 ng/mL fibroblast growth factor 2 (FGF2), 10 ng/mL vascular endothelial growth factor (VEGF) (optional), 20 ng/mL epidermal growth factor (EGF) (optional), 3 μM CHIR99021, 0.5 μM A83-01, and 50 μg/mL ascorbic acid, and incubated in the CO₂ incubator for 4 days with medium change every 2 days. The medium was then switched to advanced DMEM/F12 with 2% B27, 1% N2, 10 mM HEPES, 1% Glutamax, 1% Pen/Strep, and 2 μM retinoic acid (RA), and incubated in the CO2 incubator for 4 days with medium change every 2 days. The final media switch was to hepatocyte culture medium (HCM; Lonza, MD, USA) and the cells were incubated in a CO2 incubator for 6 days, changing the medium every 2 days.

Induction of steatohepatitis HLO (sHLO) and measurement of lipid accumulation. HLO was isolated from Matrigel and washed with 1×PBS, then cultured with HCM media containing 5 μg/ml insulin and 300 μM sodium oleate (Sigma) on ultra-low attachment 6 multi-well plates (Corning) to induce sHLO. sHLO were collected at day 3 for lipids accumulation and inflammation test at day 3 or 7. Accumulation of lipid in HLOs were measured using BODIPY® 493/503 (ThermoFisher Scientific), respectively. Briefly, sHLOs were rinsed three times with warm PBS to remove any residual oleic acid on the cell surface. Lipids accumulated in sHLOs and nuclei were stained with 2 μM BODIPY® 493/503 and NucBlue™ Live ReadyProbes™ Reagent (ThermoFisher Scientific). After staining, sHLOs were scanned using a Nikon A1 inverted confocal microscope (Japan) and Keyence BZ-X710 automated fluorescence microscope (Japan). The lipid droplet volume was calculated by using Analysis Application Hybrid cell count (Keyence) and normalized with each nucleus signal.

Donor identification and phenotypic screening of pooled organoid panels. Details of the donor identification of pooled organoid panels are described in Extended data FIG. 3. In brief, donor-specific SNP genotypes are used to detect the ratio of each donor in a multi-donor HLO panel. The gDNA of each donor-derived iPSC was extracted and the SNP profile was obtained by SNP array. Based on the SNP profile, each donor-specific SNP was selected from TaqMan SNP Genotyping Assays (Thermo Fisher Scientific Inc). Standard curves for each donor were generated using donor gDNA mixed in arbitrary ratios. The gDNA of the multi-donor HLO panel was extracted in batches and the ratio of each donor was determined using the generated standard curve. For screening of lipid accumulation phenotypes in the multi-donor HLO panel, The high and low lipid accumulation phenotype groups were separated under fluorescence microscopy from 24 donor population organoid panel of fatty acid-induced sHLO. The cutoff value of fluorescence intensity was set to 50 using BZ-X710 automated fluorescence microscope and Analysis Application Hybrid cell count (Keyence). The gDNA from the two isolated groups was extracted with DNeasy Blood & Tissue Kit (Qiagen), and the distribution of each donor-specific SNP was measured using SNP donor identification method as described.

NMR based metabolomics analysis. To obtain both polar and non-polar fractions of the HLOs for NMR analysis, all sample preparation was completed as previously described. Briefly, the appropriate volumes of solvents (final constant ratio of 2:2:1.8 of chloroform:methanol:water) were added to HLO samples. The hydrophilic extract and non-polar fraction were dried in a vacuum centrifuge at room temperature and stored at −80° C. until further preparation for NMR data collection. On the day of the data collection, dried polar extracts were re-hydrated with 220 μL of NMR buffer containing 100 mM phosphate buffer, pH7.3, 1 mM TMSP (3-Trimethylsilyl 2,2,3,3-d4 propinoate), 1 mg/mL sodium azide) prepared in D₂O. The non-polar extracts were suspended with 220 uL D-chloroform with 0.3 v/v TSP. Final volume of 200 μL of each sample was transferred into 103.5 mm×3 mm NMR tube (Bruker Biospin, Germany) for data collection. NMR spectra were recorded and processed as previously described on a Bruker Avance II 600 MHz spectrometer with BBO Prodigy probe. Metabolite assignments and quantification were performed using Chenomx® NMR Suite profiling software (Chenomx Inc. version 8.4) based on the internal standard, TMSP. The metabolites abundances were normalized to total protein prior to statistical analysis.

Measurement of insulin responsiveness in sHLO by Western Blot (WB) and gene expression analysis. HLOs and sHLOs were starved with 0.2% FCS/DMEM/F12 for 18 h and then stimulated with insulin (170 ng/ml) for 20 min. HLOs were then washed twice with PBS and lysed with M-PER™ Mammalian Protein Extraction Reagent (Thermo Fisher Scientific Inc.). Protein quantification was measured using the Pierce™ Rapid Gold BCA Protein Assay Kit (Thermo Fisher Scientific Inc.). For WB analysis, 10 μg protein were separated on 8% SDS-polyacrylamide gels, transferred to nitrocellulose membranes, and probed with anti-AKT (Clone C67E7, Cell signaling technologies, MA, USA), anti-phospho-AKT (Clone C31E5E, Cell signaling technologies), anti-α-tubulin (Clone DM1A, Cell signaling technologies) as indicated. For gene expression analysis, starved HLOs and sHLOs were treated without or with insulin for 6 h and extracted mRNA as previously described. qPCR was carried out using TaqMan gene expression master mix (Applied Biosystems) on a QuantStudio 5 Real-Time PCR System (Thermo Fisher Scientific Inc.). All primers and probe information for each target gene was shown in FIG. 18. All the results were normalized with 18S.

Measurement of GCK activity. GCK activity of HLOs was measured using the PicoProbe™ Glucokinase Activity Assay Kit (BioVision inc., CA, USA) according to manufacturer's protocol. HLOs were homogenized with 100 μl ice-cold GCK Assay Buffer containing 2.5 mM DTT and kept on ice for 10 min. The samples were centrifuged at 12,000×g at 4° C. for 10 min and the supernatant was collected. Fluorescence was measured using a BioTek™ Synergy™ H1 hybrid multi-mode monochromator fluorescence microplate reader (BioTek, VT, USA). The total protein content was also measured, and fluorescence intensity was normalized to total protein.

Metformin (MET), nicotinamide riboside (NR) and nitazoxanide (NTZ) treatments. sHLOs were cultured in HCM media in the presence or absence of 250 μg/ml MET, 1 mM NR and 3 μM NTZ. To assess the improvement of lipid accumulation in sHLO, BODIPY staining was performed. These HLOs were further assayed by reactive oxygen species (ROS) imaging and RT-qPCR.

Measurement of NAD/NADH. NAD/NADH of HLOs and sHLOs was measured using the NAD/NADH Quantitation Kit (Sigma, MO, USA) according to manufacturer's protocol. HLOs were rinsed with cold PBS, and then extracted with 500 μL of NADH/NAD Extraction Buffer by homogenization. The sample was mixed vigorously by vortexing for 30 see, and then centrifuged at 13,000×g at 4° C. for 10 minutes to remove the insoluble fraction. Fluorescence was measured using a BioTek™ Synergy™ H1 hybrid multi-mode monochromator fluorescence microplate reader (BioTek, VT, USA). The fluorescence intensity was normalized to the total protein concentration of the respective sample.

Measurement of Oxygen Consumption rate (OCR). OCR of HLOs and sHLOs was measured using the Extracellular Oxygen Consumption Assay (Abcam, Cambridge, UK) according to manufacturer's protocol. About 300 organoids were distributed into each well of black flat- and clear-bottomed 96-well microplate (Corning). Fluorescence intensity (380 nm excitation and 650 nm emission) was measured kinetically for 180 minutes using BioTek™ Synergy™-11 hybrid multi-mode monochromator fluorescence microplate reader with a time-resolved fluorescence mode. The delay time was set as 30 pts and total time of windows was 100 pts. OCR was calculated from the linear portion of the fluorescence intensity versus time plot, and then normalized to the total HLO number counted by Keyence BZ-X710 automated fluorescence microscope with cell count Analysis Application (Keyence).

Live imaging of reactive oxygen species (ROS). ROS production in HLO and sHLOs and nuclei were stained with 5 μM CellROX™ Orange Reagent, for oxidative stress detection (ThermoFisher Scientific) and NucBlue™ Live ReadyProbes™ Reagent, respectively. After staining, HLOs and sHLOs were scanned using a Nikon A1 inverted confocal microscope (Japan) and Keyence BZ-X710 automated fluorescence microscope (Japan). The ROS production was calculated by using Analysis Application Hybrid cell count and normalized with each nucleus signal.

RNA-seg data and informatics. The DonorMatched™ RNASeq Characterization Data Set (Samsara Sciences, #RSDP) were used for the four human hepatocyte transcriptome datasets (n=3 per donor). Whole-transcriptome RNA sequencing of HLOs generated from two donors of TT risk and four donors of CC non-risk (n=2 each per donor) was performed by Novogene (China) on an Illumina Novaseq S4 platform. RNA sequencing parameters were 150 bp pair-end sequencing at a depth of 20M reads per sample. Clean data were generated from the raw data that was filtered by data-processing steps, including removal of adapter sequences, reads with more than 10% N, and low-quality sequences (the percentage of low-quality bases of quality value<5 is greater than 50% in a read).

All the Fastq read files for each sample, for both human hepatocyte and HLOs were then aligned to hg19 version of the human genome using the Computational Suite for Bioinformaticians and Biologists version 3.0 (CSBB-v3.0, https://github.com/praneet1988/Computational-Suite-For-Bioinformaticians-and-Biologists) to obtain raw transcript counts. The trimmed mean of M-values (TMM) normalized Log 2 Counts-per-Million (CPM) values were obtained and analyzed for differential expression with interactive Gene Expression Analysis Kit and for Gene Set Enrichment Analysis (GSEA). For differential expression, statistical and biological significance was set at p<0.05, fold-change>1.5, Benjamini-Hochberg procedure was used for multiple testing, with a minimum of 0.5 CPM in one of the samples.

The GSKR variant was determined from the RNA-seq data using the Genome Analysis Toolkit v4.0 haplotype caller (GATK HC) after merging the triplicate data. The variants were filtered using GATK variantfilteration step and were annotated using ANNOVAR.

Curated gene sets of Reactome (https://reactome.org/), a general-purpose public database of human pathways, were used for GSEA. The pathways with significant difference of p<0.05 were selected for further analysis and ordered by normalized enrichment score (NES).

Genotyping of NASH clinical trial participants. DNA of consenting subjects enrolled in NASH clinical trials was submitted for whole genome sequencing (performed by WuXi NextCODE). Genomes were sequenced to an average coverage of 20× using paired 150 nt reads. DNA sequencing reads were aligned to the human genome (GRCh38) using BWA-MEM software. Genotypes were called for each sample using a GATK4.0-compatible computational pipeline.

IL1b and TNFa ELISA. Inflammation cytokines secretion of HLOs and sHLOs was measured using the MSD V-PLEX Proinflammatory Panel assay kit (Meso Scale Diagnostics, MD, USA) according to manufacturer's protocol. To measure secreted IL1b and TNFa, culture supernatants were collected after 72 h of culture. HLO in the analyzed wells was quantified using the CellTiter-Glo® 3D Cell Viability Assay (Promega, WI, USA) to normalize secreted cytokine.

Quantification and statistical analysis. Statistical analysis was performed using unpaired two-tailed Student's t-test, Dunn-Holland-Wolfe test, or Welch's t-test. P values<0.05 were considered statistically significant. N-value refers to biologically independent replicates. For comparisons between unpaired 2 groups, when groups were independent and the variances were unequal, non-parametric Brunner-Munzel test was performed, unless noted otherwise.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described herein without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed herein. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1. A method of making a liver organoid comprising:
a) embedding a foregut progenitor (FG) cell in a basement membrane matrix environment;
b) exposing the embedded FG cell to an FGF activator, a TGF-beta inhibitor, and a Wnt pathway activator for a period sufficient to promote expansion of the FG cell;
c) exposing the expanded FG cells of b) to a retinoic acid pathway activator for a period of time sufficient to differentiate the expanded FG cells into a liver organoid; and
d) optionally exposing the liver organoid of c) to hepatocyte growth factor (HGF), oncostatin M (OSM), dexamethasone (DEX) and insulin for a period of time.

2. A method of making a population organoid panel comprising:
a) embedding a plurality of individual progenitor cells in a single basement membrane matrix environment, wherein each of the plurality of progenitor cells is from a different donor, and
b) differentiating the plurality of progenitor cells into organoids.

3. The method of claim 2, wherein the population organoid panel is a liver organoid panel, the progenitor cells are FG cells, and differentiating the plurality of FG cells into liver organoids comprises:
a) exposing the embedded FG cells to an FGF activator, a TGF-beta inhibitor, and a Wnt pathway activator for a period sufficient to promote expansion of the FG cells to form a plurality of expanded FG cells, wherein each of the plurality of expanded FG cells of comprises cells from only a single donor;
b) exposing the plurality of expanded FG cells to retinoic acid for a period of time sufficient to differentiate them into a plurality of liver organoids, wherein each liver organoid of the plurality of liver organoids comprises cells from only a single donor; and
c) optionally exposing the liver organoids of b) to hepatocyte growth factor (HGF), oncostatin M (OSM), dexamethasone (DEX) and insulin for a period of time.

4. The method of claim 1, wherein the FGF activator is FGF2, optionally in an amount of 0.5-50 ng/ml, 1-25 ng/mL, 2.5-10 ng/mL, or 5 ng/mL.

5. The method of claim 1, wherein the TGF-beta inhibitor is A83-01, optionally in an amount of 0.05-5.0 μM, 0.1-2.5 μM, 0.25-1.0 μM, or 0.5 μM.

6. The method of claim 1, wherein the Wnt pathway activator is a GSK-3 inhibitor, optionally CHIR99021, optionally in an amount of 0.3-30 μM, 0.6-15 μM, 1.5-6 μM, or 3 μM.

7. The method of claim 1, wherein the retinoic acid pathway activator is retinoic acid, optionally in an amount of 0.2-20 PM, 0.4-10 PM, 1.0-4 μM, or 2 μM.

8. The method of claim 1, wherein the period sufficient to promote expansion of the FG cell to form expanded FG cells is about 1-8, 2-6, 3-5, or 4 days.

9. The method of claim 1, wherein the period of time sufficient to differentiate the expanded FG cells into a liver organoid is about 1-8, 2-6, 3-5, or 4 days.

10. The method of claim 1, further comprising culturing the liver organoid in hepatocyte culture medium.

11. The method of claim 1, wherein the FG cell is differentiated from an induced-pluripotent stem cell (IPSC) by exposure to Activin A and optionally a BMP pathway activator, a Wnt pathway activator, and an FGF activator.

12. The method of claim 11, wherein the IPSC is exposed to Activin A and optionally a BMP pathway activator, optionally BMP4, for a first period of time, optionally about 1-4, 2-4, or 3 days, and then exposed to the Wnt pathway activator and an FGF activator for a second period of time, optionally about 1-4, 2-4, or 3 days.

13. The method of claim 11, wherein the method comprises dissociating clusters of FG cells into single FG cells, and optionally, cryopreserving single FG cells.

14. The method of claim 11, wherein:
the Activin A is in an amount of 1-10000 ng/mL, 10-1000 ng/mL, 20-500 ng/mL, 50-200 ng/mL, or 100 ng/mL,
the Wnt pathway activator is a GSK-3 inhibitor, optionally CHIR99021, optionally in an amount of 0.3-30 μM, 0.6-15 μM, 1.5-6 μM, or 3 μM; and/or
the FGF activator is FGF4, optionally in an amount of 50-5000 ng/ml, 100-2500 ng/mL, 250-1000 ng/mL, or 500 ng/mL.

15. The method of claim 2, wherein the plurality of individual FG cells are from at least 20 different donors.

16. The method of claim 2, wherein the liver organoid has a genotype comprising a single nucleotide polymorphism (SNP) variant selected from the group consisting of PNPLA3 rs738409, GCKR-rs1260326, GCKR-rs780094, and TM6SF2-rs58542926.

17. The method of claim 1, wherein the FG cell is human.

18. The method of claim 1, further comprising exposing the liver organoid to a fatty acid, optionally oleic acid, and optionally insulin, to generate a steatohepatitis-like liver organoid.

19. A method of determining a genotype associated with a non-alcoholic fatty liver disease (NAFLD) or non-alcoholic steatohepatitis (NASH) phenotype, comprising
a) generating a population liver organoid panel with steatohepatitis-like liver organoids by the method of claim 18;
b) maintaining the population liver organoid panel in a shared environmental condition;
c) observing the phenotype of an individual steatohepatitis-like organoid;
d) sequencing a nucleic acid sample of the individual steatohepatitis-like clonal organoid to the identify a genotype of the individual steatohepatitis-like clonal organoid; and
e) correlating the genotype of the individual steatohepatitis-like clonal organoid with the observed phenotype of the individual steatohepatitis-like clonal organoid.

20. The method of claim 3, further comprising administering a compound of interest to the population liver organoid panel.

21. The method of claim 20, wherein the compound of interest is selected based on a correlated genotype-phenotype of the steatohepatitis-like clonal organoid.

22. The method of claim 20, further comprising assessing the efficacy of the compound of interest for treating NAFLD/NASH by observing a response of the individual steatohepatitis-like clonal organoid to the compound.

23. The method of claim 20, wherein the compound of interest is PFK15, AMG3969, metformin, nitazoxanide (NTZ), and/or nicotinamide riboside (NR).

24. The method of claim 19, wherein the shared environmental conditions are insulin insensitive.

25. The method of claim 24, wherein the insulin insensitive conditions comprise high glucose and/or high insulin culture conditions.

26. The method of claim 19, wherein the observed phenotype is lipid accumulation, inflammation and/or mitochondrial function.

27. The method of claim 19, wherein the genotype is a single nucleotide polymorphism (SNP) variant.

28. The method of claim 27, wherein the single nucleotide polymorphism (SNP) variant is PNPLA3 rs738409, GCKR-rs1260326, GCKR-rs780094, or TM6SF2-rs58542926.

29. A liver organoid made by the method of claim 1.

30. A liver population organoid panel made by the method of claim 3.

31. The liver population organoid panel of claim 30, wherein the organoids are liver organoids and/or steatohepatitis-like liver organoids.

32. A liver organoid having a SNP variant selected from the group consisting of PNPLA3 rs738409, GCKR-rs1260326, GCKR-rs780094, and TM6SF2-rs58542926.

33. A method of screening a compound of interest comprising administering a compound of interest to the liver organoid of claim 29.

34. The method of claim 33, wherein the compound of interest is selected based on a correlated genotype-phenotype of the steatohepatitis-like clonal organoid.

35. A method comprising:
a) obtaining or having obtained the HbA1c level of a subject identified as having a SNP variant GCKR-rs1260326;
b) determining that the subject has a reduced risk of and/or a good prognosis for fatty acid liver disease if the subject's HbA1c level is less than 5.7%, or c) determining that the subject has an increased risk of and/or a poor prognosis for fatty acid liver disease if the subject's HbA1c level is greater than 6.4%.

36. A method for treating a subject identified as having a SNP variant GCKR-rs1260326, the method comprising:
a) obtaining or having obtained the HbA1c level of the subject identified as having a SNP variant GCKR-rs1260326;
b) determining that the subject has a reduced risk of and/or a good prognosis for fatty acid liver disease if the subject's HbA1c level is less than 5.7%, or determining that the subject has an increased risk of and/or a poor prognosis for fatty acid liver disease if the subject's HbA1c level is greater than 6.4%; and
c) administering to the subject that has an increased risk of fatty acid liver disease having an HbA1c level greater than 6.4% a treatment that results in oxidative uncoupling.

37. A method for treating a subject comprising:
selecting a subject identified as having a SNP variant GCKR-rs1260326 and HbA1c level greater than 6.4%; and
administering to the subject a treatment that results in oxidative uncoupling.

38. The method of claim 36, wherein treatment that results in oxidative uncoupling comprises a NAD+ precursor, optionally nicotinamide riboside, and nitazoxanide.

39. The method of claim 36, wherein treatment further comprises metformin.

40. The method of claim 35, wherein the fatty acid liver disease is NAFLD and/or NASH.
